(19) United States
(12) Patent Application Publication
(10) Pub. No.: US 2016/0243167 A1
(43) Pub. Date: Aug. 25, 2016

(54) COMPOSITIONS AND METHODS FOR MESENCHYMAL/STROMAL STEM CELL REJUVENATION AND TISSUE REPAIR BY ENHANCED CO-EXPRESSION OF TELOMERASE AND MYOCARDIN

(71) Applicants: Yong-Jian GENG, Houston, TX (US); Rosalinda MADONNA, Houston, TX (US); James T. Willerson, Houston, TX (US)

(72) Inventors: Yong-Jian GENG, Houston, TX (US); Rosalinda MADONNA, Houston, TX (US); James T. Willerson, Houston, TX (US)

(21) Appl. No.: 14/995,061

(22) Filed: Jan. 13, 2016

Related U.S. Application Data
(63) Continuation of application No. 13/975,012, filed on Aug. 23, 2013, now abandoned.
(60) Provisional application No. 61/693,034, filed on Aug. 24, 2012.

(51) Int. Cl. A61K 35/28; C12N 15/85; A61K 9/00

(52) U.S. Cl. CPC A61K 35/28; A61K 9/0014; C12N 15/85

(57) ABSTRACT

A major risk factor for ischemic heart disease is advanced age. In adult bone marrow and other tissues, the number and function of stem cells decline with aging. Telomerase reverse transcriptase (TERT) is a nuclear protein that decreases senescence. Myocardin (MYOCD) is a transcription factor for myogenesis. Thus a method is provided for the simultaneous delivery of the telomerase reverse transcriptase (TERT) and myocardin MYOCD genes that resuscitates mesenchymal stromal cells (MSCs) from aged adipose and bone marrow tissues by increasing their capacity for survival, proliferation, and differentiation. TERT⁺/MYOCD⁺ MSCs restores a capacity for repairing ischemic tissues via improved blood flow and revascularization.

### CROSS-REFERENCE TO RELATED APPLICATION

This U.S. continuation patent application claims the benefit of priority to U.S. patent application Ser. No. 13/975,012 filed on Aug. 23, 2013 and U.S. Provisional Patent Application No. 61/693,034, filed Aug. 24, 2012, the disclosures of which are hereby incorporated herein by reference in their entirety.

### STATEMENT REGARDING FEDERALLY SPONSORED RESEARCH OR DEVELOPMENT

This invention was made with government support under Grant Nos. R01HL59249, R01HL69509 and W81×WH-04-2-0035 from the United States Department of Defense.

### BACKGROUND

Patients with severe obstructive vascular disease, usually caused by atherosclerotic plaque narrowing of arteries are often aged and have tissue resident and circulating vascular stem/progenitor cells with diminished functions^{1, 2}. These functional deficits may cause a poor angiogenic response to hypoxia or ischemia, with impaired collateral vessel formation and microcirculation³. Likewise with age, which is a major risk factor for cardiovascular disease, regenerative properties deteriorate and consequently resident stem/progenitor cells in elderly humans may have a decreased capacity for repair in response to tissue injury. Also in aged tissues, myogenic or angiogenic stem cells may transform into fibroblasts which contribute to enhanced fibrosis⁴⁻⁶. These combined age-related deficits likely contribute to decreased muscle, and vessel regeneration after injury and facilitation of atherosclerosis and its sequelae in older individuals^{7, 29}. Replenishing stem cell function either by rejuvenating existing aging cells or transplanting stem/progenitor cells from donors capable of supplying the ischemic tissue with new vessels and preventing ischemic tissue damage have been considered an appropriate therapy for this condition⁸⁻¹⁰.

### BRIEF SUMMARY OF THE DISCLOSED EMBODIMENTS

The present invention provides methods and compositions for the diagnosis of stem cell senescence by assessing co-expression and interaction between the two nuclear proteins, Telomerase Reverse Transcriptase (TERT) and myocardin (MYOCD), and for the rejuvenation of aging or senescent stem cells from the mesenchymal or stromal compartments of mammalian tissues or organs by simultaneous delivery of the TERT and MYOCD genes. Aging and diseased stem cells express low levels of TERT and MYOCD. The resuscitation of stem cells, such as mesenchymal stromal cells (MSCs) from aged tissues or organs, including but not limited to adipose and bone marrow tissues. increases the stem cell capacity for survival, proliferation, and differentiation. The methods for assessing co-expression and interaction between TERT and MYOCD are useful for evaluation of the stem cell senescence in aged or diseased individuals. The TERT/MYOCD rejuvenated stem cells possess high levels of potency of growth and differentiation and are capable of repairing ischemic tissues via improved blood flow and revascularization, and in some embodiments may be used as diagnostic and/or therapeutic agents. Hence, In some embodiments of the present invention, a method is provided for rejuvenating MSCs and increasing their therapeutic efficacy in regenerating or repairing tissues of mammalian hearts and blood vessels damaged by infarction or short of blood supply (ischemia), the method comprises isolating MSCs cloning cDNA coding for the catalytic unit of telomerase or TERT and the nuclear promyogenic transcriptional factor MYCOD into an expression vector, such as plasmids and lentivirus and thereby producing lentiviral vectors comprising TERT and MYCOD genes; transducing said MSCs with said TERT/MYCOD-carrying vectors, thereby forming genetically modified MSCs that have increased expression of TERT and MYCOD; and repairing tissue by administering to said tissues TERT/MYCOD-tranduced MSCs, wherein said TERT/MYCOD-tranduced MSCs display an increase in at least one of survival, proliferation, and differentiation as compared to MSCs that do not co-express TERT and MYCOD at a significant level.

In one embodiment of the method of assessing the senescence of stem cells from aged individuals or those with age-associated diseases, such as atherosclerosis, expression and interaction of TERT and MYOCD are identified by molecular fluorescent resonance and immunoprecipitation. Stem cells with compromised expression of TERT/MYOCD predict poor capacity of tissue regeneration and repair, and the need for rejuvenation.

In another embodiment of the method of rejuvenating mesenchymal stromal cells, said administering further increases blood flow and revascularization of said tissue. In a further embodiment of the method of rejuvenating mesenchymal stromal cells, MSCs are isolated from adult tissues, including but not limited to the adipose and bone marrow tissues, in a further embodiment the MSCs are derived from a mammalian tissue, including but not limited to murine or human tissues, and in a still further embodiment the isolated MSCs are adult and aged.

In one embodiment of the method of rejuvenating mesenchymal stromal cells described herein, said TERT and MYOCD cDNAs are full-length with all coding sequences, and they are inserted into said lentiviral vector that is a pLenti-TOPO-type cloning vector. In another embodiment of the method of rejuvenating mesenchymal stromal cells, said administering comprises at least one intramuscular injection, and in a further embodiment, the at least one intramuscular injection comprises at least 3×10⁶ TERT/MYOCD-transduced MSCs.

In some embodiments of the method of rejuvenating mesenchymal stromal cells, said increased expression prevents cytotoxic cell death, as compared to MSCs that do not over express TERT and MYOCD, in a further embodiment said increased expression increases resistance to Fas induced and Non-Fas induced apoptosis, as compared to MSCs that do not over express TERT and MYOCD. In another embodiment, said increased expression increases the differentiation potential to develop into mesenchymal cell lineages, including but not limited to cardiomyocytes, smooth muscle cells and bone-forming cells, as compared to MSC's that do not over express TERT and MYOCD. In a further embodiment said overexpression decreases adipogenic differentiation potential of MSC as compared to that of MSC, which do not over express TERT and MYOCD.

In some embodiments of the method of rejuvenating mesenchymal stromal cells, said MSCs with enhancement of TERT/MYOCD expression increase arteriogenisis as compared to MSCs that do not over express TERT and MYOCD. In another embodiment of the method of rejuvenating mesenchymal stromal cells described herein, transducing said MSCs further comprises incubating said isolated MSCs with media containing TERT/MYCOD-inserted vectors for about 16 hrs in polybrene, and in a further embodiment said TERT/MYCOD-transduced MSCs are further propagated by culturing in a culture medium for about 5 days.

In another embodiment a method of treating an individual suffering from a cardiovascular condition is herein described, wherein said method comprises administering to said individual TERT and MYCOD co-tranduced mesenchymal stromal cells (MSC), wherein said transduced cells have enhanced expression of TERT and MYCOD; and wherein said the increased TERT and MYCOD co-expression increases survival, proliferation and differentiation of said MSCs. In a further embodiment of the method of treating an individual suffering from a cardiovascular condition, the MSC's are autologous or allogeneic MSCs from adult tissues, including but not limited to adipose and bone marrow tissues, administering said MSCs further increases blood flow, revascularization, and repair of damaged tissue comprising said pathological conditions. In another embodiment a method of propagating adult stem cells with enhanced expression of Telomerase Reverse Transcriptase (TERT) and Myocardin (MYCOD) is described herein, the method comprises isolating MSCs; cloning TERT and MYCOD in lentiviral expression plasmids thereby producing lentiviral vectors comprising TERT and MYCOD genes; transducing said MSCs with said lentiviral vectors, thereby forming lentivirus-tranduced MSCs wherein said transduced MSCs over-express TERT and MYCOD; and propagating said TERT/MYCOD-tranduced MSCs by maintaining said transduced MSCs in culture for about 5 days post transduction. In a further embodiment a method of constructing a viral vector that carries cDNAs coding for the full-length coding sequences of TERT and MYCOD is herein described, the method comprises cloning of TERT and MYCOD in lentiviral expression plasmids; wherein said cloning comprises, amplifying full-length cDNAs for human TERT and full-length cDNAs for human MYOCD by PCR; and subcloning and expressing the cDNAs into the pLenti-TOPO cloning vector to produce a lentviral vector that is capable of co-expressing mycocardin and telomerase cDNA. In a further embodiment a composition for repairing ischemic tissue is herein described, the composition comprises a plurality of lentivirus-tranduced mesenchymal stromal cells (MSCs) wherein said lentivirus-transduced cells over-express Telomerase Reverse Transcriptase (TERT) and Myocardin (MYCOD); and a pharmaceutically acceptable carrier.

### BRIEF DESCRIPTION OF THE DRAWINGS

For a detailed description of exemplary embodiments of the invention, reference will now be made to the accompanying drawings.

FIG. 1: Aged adipose mesenchymal stromal cells (MSCs) with enhanced expression of TERT and MYOCD become highly clonogenic. Clonogenic assessment of MSCs overexpressing TERT and MYOCD (n=5 mice per each group). The colony appearance of adipose MSCs harvested from aged (12-months old) and young (1 month old) C57 or ApoE^{-/-} mice was detected up to day 15. Panels show phase-contrast photomicrographs of the colony appearance of MSCs in methylcellulose. Magnification is 5×. Images are representative of 3 independent experiments.

FIG. 2A-2B: Decreased total cell death and apoptotic response to Fas induction in aged bone marrow mesenchymal stromal cells (MSCs) overexpressing TERT and MYOCD. Cytotoxicity and apoptosis assays. FIG. 2A, Representative images show quantification of total cell death as a function of TERT and MYOCD transduction (n=5 mice per each group). Sytox staining of mock- or TERT- or MYOCD-transduced MSCs harvested from bone marrow of aged C57 mice (12 months old) was assessed by flow cytometry. Graphs represent combined data from 3 independent experiments; results are the mean±SD. FIG. 2B, Representative images show survival effects as a function of TERT and MYOCD transduction (n=5 mice per each group). Evidence of apoptosis by staining for Annexin-V, propidium iodide, or both is reduced in TERT or MYOCD-overexpressing MSCs. Annexin-V/propidium iodide (PI) staining of mock- or TERT- or MYOCD-transduced MSCs, harvested from bone marrow of aged C57 mice (12 months old), in the presence or absence of Fas/CD95 (500 ng/mL) treatment was assessed by flow cytometry. The results are representative of 3 independent experiments. Quadrants are defined as follows: live (lower left), necrotic (lower right, Q1), apoptotic-necrotic (upper right, Q2) and apoptotic (lower right, Q4). Images are representative of 3 independent experiments.

FIG. 3: Increased rate of osteogenic differentiation in aged adipose mesenchymal stromal cells (MSCs) with enhanced expression of TERT and MYOCD. Representative images show mineralization as a function of TERT and MYOCD transduction (n=5 mice per each group) in MSCs harvested from aged (12 months old) and young (1 month old) C57 or ApoE^{-/-} mice. The degree of mineralization was determined in 12-well plates using Alizarin Red staining and normalized to the relative number of viable cells as determined directly in 96-well plates as described in “Materials and Methods.” Graphs represent combined data from 3 independent experiments; results are the mean±SD. Magnification is 10×.

FIG. 4: Decreased rate of adipogenic differentiation in aged adipose mesenchymal stromal cells (MSCs) with enhanced expression of TERT and MYOCD. Representative images show lipid accumulation as a function of TERT and MYOCD transduction (n=5 mice per each group) of adipose MSCs harvested from aged (12 months old) or young (1 month old) C57 mice. The degree of adipocyte differentiation was determined in the 12-well plates using Oil Red O staining and normalized to the relative number of viable cells as determined directly in the 96-well plates as described in “Materials and Methods.” Graphs represent combined data from 3 independent experiments; results are mean±SD. Magnification is 5× and 10×.

FIG. 5: Decreased rate of adipogenic differentiation in aged adipose mesenchymal stromal cells (MSCs) with enhanced expression of TERT and MYOCD. Representative images show lipid accumulation as a function of TERT and MYOCD transduction (n=5 mice per each group) of adipose MSCs harvested from aged (12 months old) or young (1 month old) ApoE^{-/-} mice. The degree of adipocyte differentiation was determined in the 12-well plates using Oil Red O staining and normalized to the relative number of viable cells as determined directly in the 96-well plates as described in “Materials and Methods.” Graphs represent combined data from 3 independent experiments; results are mean±SD. Magnification is 5× and 10×.

FIG. 6A-6B: TERT and MYOCD overexpression enhances cardio-myogenesis in aged mesenchymal stromal cells (MSCs). FIG. 6A, western analysis of cardiac actin and smooth-muscle α-actin expression in mock- or TERT- or MYOCD-transduced MSCs harvested from adipose tissue (AT-MSCs) or bone marrow (BM-MSCs) of aged C57 mice (12 months old, n=5 mice per each group). Control analyses were done by using human bone marrow mesenchymal stem cells (hMSCs). The proteins were stripped and re-incubated with GAPDH. FIG. 6B, densitometric analysis, results are representative of three different experiments, data represent mean±SD, *p<0.05 and **p<0.01 versus mock-transduced.

FIG. 7A-7B: TERT and MYOCD overexpression enhances blood flow in aged hypercholesterolemic mice after hindlimb ischemia. Therapeutic efficiency of mock- or TERT- or MYOCD-transduced GFP⁺ MSCs in murine model of hindlimb ischemia. LDPI performed at day 28 after administration of transduced GFP⁺ MSCs. FIG. 7A, Impact on hindlimb integrity. FIG. 7B shows:Administration of GFP⁺ MSCs increased blood flow compared with that of saline-injected controls. Compared with mice transplanted with mock-transduced GFP⁺ MSCs, mice transplanted with TERT and MYOCD-transduced GFP⁺ MSCs demonstrated enhanced perfusion measured by LDPI. Inserts: mock-transduced GFP⁺ MSCs or TERT and MYOCD-transduced GFP⁺ MSCs.

FIG. 8A-8D: TERT and MYOCD overexpression enhances arteriogenesis in aged hipercholesterolemic mice after hindlimb ischemia. Histologic evidence of arteriogenesis in ischemic hindlimb. Representative photomicrographs of capillaries (panels FIG. 8A and FIG. 8B) and arterioles (panels FIG. 8C and FIG. 8D) in tissue sections from muscle of ischemic legs stained with alkaline phosphatase. The extent of neovascularization was assessed by measuring arterioles density in light microscopic sections prepared from muscles of ischemic hindlimbs. Arterioles density was significantly greater in hindlimbs of mice receiving TERT and MYOCD-transduced GFP⁺ MSCs compared with mock-transduced GFP⁺ MSCs, and in hindlimbs of mice receiving mock-transduced GFP⁺ MSCs compared with saline.

FIG. 9A-9D, shows Fluorescence-activated cell sorting (FACS) and western analysis of transduced cells.

FIG. 10A-10B, show TERT- and MYOCD-transduced MSCs engraft into ischemic tissue and differentiate into vascular structures.

FIG. 11A-11B, shows multispectral imaging of transverse leg sections stained for ASMA revealed vascular differentiation of transplanted GFP+ MSCs in ischemic legs at 21 days.

FIG. 12A-12C shows co-immunoprecipitation of TERT and MYOCD in Dil-acLDL receptor positive and negative MSCs with TERT activities. (Co-immunoprecipitation of TERT and MYOCD in Dil-acLDL receptor negative and positive adipose MSCs with telomerase activities determined by TRAP assays. FIG. 12A (upper panel), Proteins pulled down with anti-TERT were immunoblotted with anti-McA and anti-TERT antibody. Immunoprecipitants from non-sorted MSCs (lane 1), Dil-acLDL⁺ MSCs (lane 2), and Dil-acLDL⁻ MSCs (lane 3). A (lower panel), Western blot for normal IgG in the same immunoprecipitated samples shown in the upper panel. FIG. 12B, Telomeric repeat amplification protocol (TRAP) assay for telomerase activity in Dil-acLDL⁺ adipose tissue-derived MSCs, DiD-acLDL⁻ adipose tissue-derived MSCs, and total adipose tissue-derived MSCs. Telomerase activity was assessed in cell lysates from total MSCs, Dil-acLDL⁺ cells, Dil-acLDL⁺ cells and culture medium only. The TRAP gel image shows typical ladders of PCR-amplified telomeric repeats, and is representative of 3 separate experiments. FIG. 12C, Quantification of telomerase activity by fluorometry. The results are representative of three separate experiments; data represent mean±standard deviation.

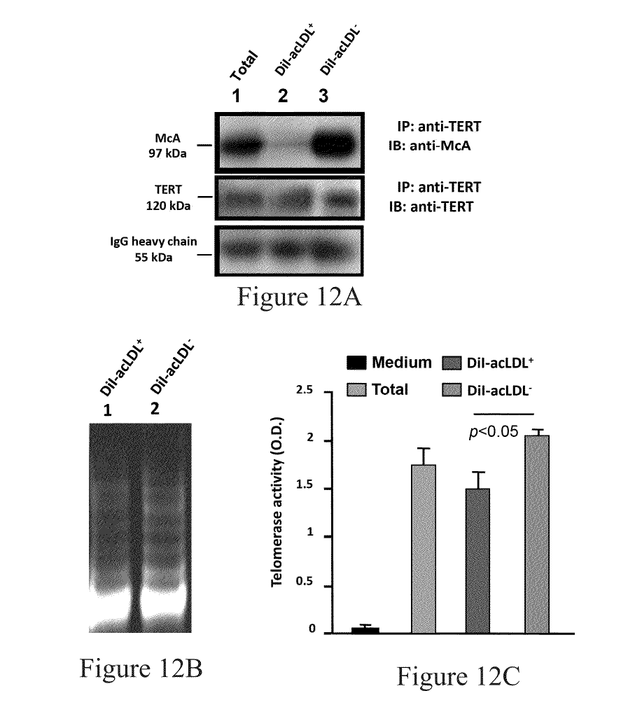

FIG. 13A-13B shows interaction between TERT and MYOCD in MSCs transfected with TERT and MYOCD cDNA as determined by Bioluminescence Resonance Energy Transfer (BRET) assays. (BRET analysis of interaction between TERT and MYOCD in adult MSCs from adipose tissues. FIG. 13A, BRET levels measured in murine MSCs transfected with combinations of donor and acceptor constructs. MSCs were transfected with RLuc-McA (MYOCD) alone or in combination with pAcGFP-TERT and subjected to the Bioluminescence Resonance Energy Transfer (BRET) assay (see Methods). In parallel experiments, MSCs were co-transfected with serial amount of pAcGFP and RLuc given as positive control for the BRET signal. Values are means±S.D. of triplicate experiments. FIG. 13B, representative BRET saturation curve, showing specificity of BRET interactions between MYOCD and TERT. Cotransfections were performed with increasing amounts of plasmid DNA for the pAcGFP-TERT construct (1, 4, and 10 μg), whereas the Rluc construct was kept constant (4 μg). Total plasmid DNA was kept constant with empty vector (pcDNA3.1). All samples were subjected to luminescence analysis and relative fluorescence units (RFU) values were plotted as a fraction of relative luciferase units (RLU). All values are expressed as mean±standard deviation from three independent experiments).

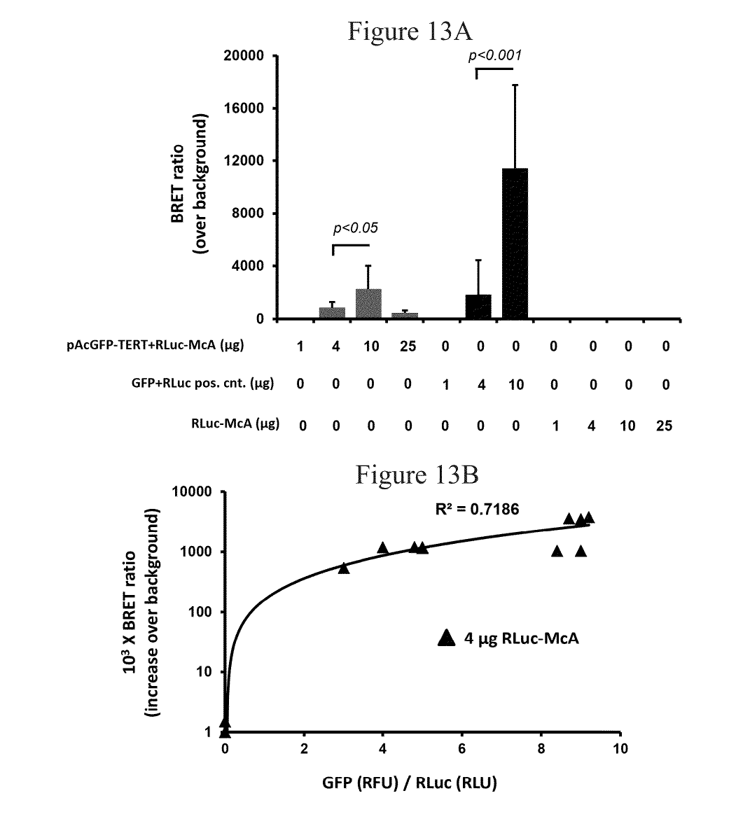

FIG. 14 shows phenotypic characterization of TERT/MYOCD EXPRESSING MSCs at low (young) and high (old) passages from adult adipose tissue of wild type and cloned pigs by flow cytometry. Flow cytometry of surface biomarkers in MSCs derived from adipose tissue of wild type (WT) and cloned pigs. (a-e), WT MSCs at low passages; (f-j), Cloned MSCs at lower passages; (k-o), WT MSCs at high passages; (p-t), Cloned MSCs at high passages. The following antibodies were used for the biomarker assessment by flow cytometry: anti-CD29, CD44, and CD90 positive, CD45, and vWF antibodies.

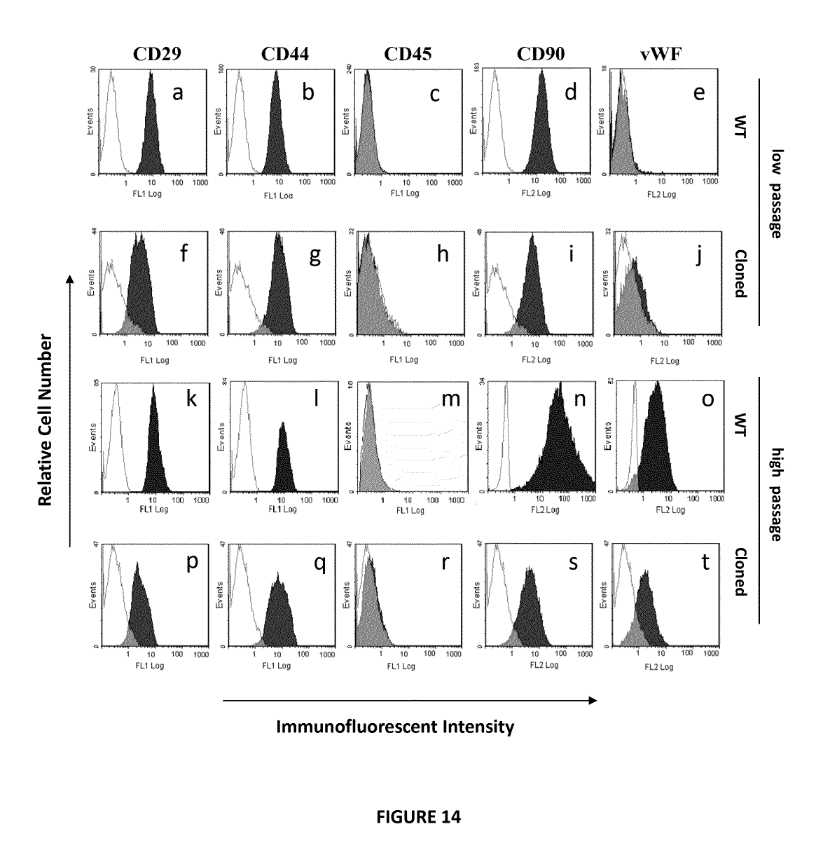

### NOTATION AND NOMENCLATURE

Certain terms are used throughout the following description and claims to refer to particular system components. As one skilled in the art will appreciate, companies may refer to a component by different names. This document does not intend to distinguish between components that differ in name but not function. In the following discussion and in the claims, the terms “including” and “comprising” are used in an open-ended fashion, and thus should be interpreted to mean “including, but not limited to . . . .” Also, the term “couple” or “couples” is intended to mean either an indirect or direct electrical connection. Thus, if a first device couples to a second device, that connection may be through a direct electrical connection, or through an indirect electrical connection via other devices and connections. In the following discussion and in the claims, the terms “about” represents ±10% of a numerical value, for example wherein a claim reads on “about 80 gm” infact claims a range of 80 gm,±8 gmDETAILED DESCRIPTION

The following discussion is directed to various embodiments of the invention. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to intimate that the scope of the disclosure, including the claims, is limited to that embodiment. Further, references cited and disclosed herein are expressly incorporated by reference in their entirety.

In some embodiments of the present invention, a method is provided for rejuvenating mesenchymal stromal cells (MSCs) and increasing their therapeutic efficacy in regenerating or repairing tissues of mammalian hearts and blood vessels damaged by infarction or short of blood supply (ischemia), the method comprises isolating MSCs cloning cDNA coding for the catalytic unit of telomerase or Telomerase Reverse Transcriptase (TERT) and the nuclear promyogenic transcriptional factor Myocardin (MYCOD) into an expression vector, such as plasmids and lentivirus and thereby producing lentiviral vectors comprising TERT and MYCOD genes; transducing said MSCs with said TERT/MYCOD-carrying vectors, thereby forming genetically modified MSCs that have increased expression of TERT and MYCOD; and repairing tissue by administering to said tissues TERT/MYCOD-tranduced MSCs, wherein said TERT/MYCOD-tranduced MSCs display an increase in at least one of survival, proliferation, and differentiation as compared to MSCs that do not co-express TERT and MYCOD at a significant level. In another embodiment of the method of rejuvenating mesenchymal stromal cells, said administering further increases blood flow and revascularization of said tissue. In a further embodiment of the method rejuvenating mesenchymal stromal cells, MSCs are isolated from adult tissues, including but not limited to the adipose or bone marrow tissues, in a further embodiment the MSCs are derived from a mammalian tissue, including but not limited to murine or human tissues, and in a still further embodiment the isolated MSCs are adult and aged.

In one embodiment of the method of rejuvenating mesenchymal stromal cells described herein, said TERT and MYOCD cDNAs are full-length with all coding sequences, and they are inserted into said lentiviral vector that is a pLenti-TOPO-type cloning vector. In another embodiment of the method of rejuvenating mesenchymal stromal cells, said administering comprises at least one intramuscular injection, and in a further embodiment, the at least one intramuscular injection comprises at least 3×10⁶ TERT/MYOCD-transduced MSCs.

In some embodiments of the method of rejuvenating mesenchymal stromal cells said increased expression prevents cytotoxic cell death, as compared to MSCs that do not over express TERT and MYOCD, in a further embodiment said increased expression increases resistance to Fas induced and Non-Fas induced apoptosis, as compared to MSCs that do not over express TERT and MYOCD. In another embodiment, said increased expression increases the differentiation potential to develop into mesenchymal cell lineages, including but not limited to cardiomyocytes, smooth muscle cells and bone-forming cells, as compared to MSC's that do not over express TERT and MYOCD, In a further emdodiment said overexpression decreases adipogenic differentiation potential of MSC as compared to that of MSC, which do not over express TERT and MYOCD.

In some embodiments of the method of rejuvenating mesenchymal stromal cells, said MSCs with enhancement of TERT/MYOCD expression increase arteriogenisis as compared to MSCs that do not over express TERT and MYOCD. In another embodiment of the method of rejuvenating mesenchymal stromal cells described herein,

transducing said MSCs further comprises incubating said isolated MSCs with media containing TERT/MYCOD-inserted said vectors for about 16 hrs in polybrene, and in a further embodiment said TERT/MYCOD-transduced MSCs are further propagated by culturing in a culture medium for about 5 days.

In another embodiment a method of treating an individual suffering from a cardiovascular condition is herein described, wherein said method comprises administering to said individual TERT and MYCOD co-tranduced mesenchymal stromal cells (MSC), wherein said transduced cells have enhanced expression of TERT and MYCOD; and wherein said the increased TERT and MYCOD co-expression increases survival, proliferation and differentiation of said MSCs. In a further embodiment of the method of treating an individual suffering from a cardiovascular condition, the MSC's are autologous or allogeneic MSCs from adult tissues, including but not limited to adipose and bone marrow tissues, administering said MSCs further increases blood flow, revascularization, and repair of damaged tissue comprising said pathological conditions. In another embodiment a method of propagating adult stem cells with enhanced expression of Telomerase Reverse Transcriptase (TERT) and Myocardin (MYCOD) is described herein, the method comprises isolating MSCs; cloning TERT and MYCOD in lentiviral expression plasmids thereby producing lentiviral vectors comprising TERT and MYCOD genes;transducing said MSCs with said lentiviral vectors, thereby forming lentivirus-tranduced MSCs wherein said transduced MSCs over-express TERT and MYCOD; and propagating said TERT/MYCOD-tranduced MSCs by maintaining said transduced MSCs in culture for about 5 days post transduction. In a further embodiment a method of constructing a viral vector that carries cDNAs coding for the full-length coding sequences of TERT and MYCOD is herein described, the method comprises cloning of TERT and MYCOD in lentiviral expression plasmids; wherein said cloning comprises, amplifying full-length cDNAs for human TERT and full-length cDNAs for human MYOCD by PCR; and subcloning and expressing the cDNAs into the pLenti-TOPO cloning vector to produce a lentviral vector that is capable of co-expressing mycocardin and telomerase cDNA. In some embodiments, TERT/myocardin increases telomerase activities, increases the length of telomeres, and slows down the aging process in MSCs from aged adults. In some further embodiments, TERT/myocardin transduction enhances myocardin activities, increases the expression of promyogenic genes, and enables said MSCs committed to differentiate into cardiovascular cells.

In another embodiment a composition for repairing ischemic tissue is herein described, the composition comprises a plurality of lentivirus-tranduced mesenchymal stromal cells (MSCs) wherein said lentivirus-transduced cells over-express Telomerase Reverse Transcriptase (TERT) and Myocardin (MYCOD); and a pharmaceutically acceptable carrier.

Therefore, in some embodiments of the present invention, Mesenchymal stromal cells derived from adipose tissue (AT-MSCs) contain a population of adult multipotent mesenchymal stem cells and endothelial progenitors that can regenerate damaged cardiovascular tissues^{10-15.} In some embodiments of the current invention a subpopulation of AT-MSCs that expresses high levels of the catalytic subunit of telomerase, telomerase reverse transcriptase (TERT), and myocardin (MYOCD)^{14, 16}, a key regulator of cardiovascular myogenic development^{15, 17, 18} has been identified.

In some embodiments, MYOCD acts as a nuclear transcription cofactor for myogenic gene transcription, muscle regeneration, and protection against apoptosis^{19, 20}, while telomerase maintains the telomere length, cell survival, and proliferation, and prevents cellular senescence^{21, 22}. In some further embodiments, it was observed that AT-MSCs with co-expression of TERT and MYOCD have increased levels of octamer-binding transcription factor 4 (Oct-4), MYOCD, myocyte specific enhancer factor 2c (Mef2c), and homeobox protein NKx2.5 indicates that key transcription factors can be induced in these cells by TERT and MYOCD transduction, thus TERT and MYOCD may act together to enhance cardiovascular myogenic development^{16, 23}.

In one embodiment, AT-MSCs, increased co-expression of the nuclear proteins, MYOCD and TERT, rejuvenated AT-MSCs promyogenic stem cells with an augmented capacity for proliferation and myogenic differentiation. In a further embodiment, MYOCD and TERT may be found to interplay or synergize to rejuvenate and promote cardiovascular myogenesis in aged MSCs. Therefore, in one embodiment, the gene delivery to increase the expression of the catalytic subunit of TERT and MYOCD impacts survival, growth, and differentiation in aged AT-MSCs, which was assessed by analyzing the therapeutic efficacy of mouse AT-MSCs in which MYOCD and TERT were overexpressed in restoring blood flow and promoting vascularization in a hypercholesterolemic mouse (ApoE−/−) model of hindlimb ischemia.

In some embodiments, TERT-positive MSCs derived from adipose tissue express receptors for acetylated low lipoprotein (acLDL), a biomarker for endothelial cells and mononuclear phagocytes. MSCs with or without fluorescently labeled acLDL were identified by flow cytometry and sorted for the assessment of TERT/MYOCD expression, myogenesis, and angiogenesis.

In another embodiment, increased expression and interaction of TERT and MYOCD in aged adult MSCs could be achieved by the delivery of TERT and MYOCD cDNAs, which facilitate the cell survival and cardiomyogenic differentiation. The interaction between TERT and MYOCD in MSCs transfected with cDNAs coding for TERT and MYOCD fused to green fluorescent protein and luciferase, respectively, could be assessed by the bioluminescence Resonance Energy Transfer (BRET) assays.

In another embodiment, phenotypic development of TERT/MYOCD EXPRESSING MSCs was characterized by flow cytometry. TERT⁺/MYOCD⁺ MSCs from normal wild type (WT) and cloned pigs were CD29⁺, CD44⁺, CD90⁺ but CD45⁻ and vWF⁻.

Methods.

Animal Care. All procedures were approved by Institutional Ethics Committee for animal research. All studies conform to either the Guide for the Care and Use of Laboratory Animals published by the United States National Institutes of Health or the Directive 2010/63/EU of the European Parliament.

Isolation of MSCs And Cell Culture. After anesthesia with isofluorane inhalation (2-5% isofluorane in oxygen), male C57/BL6 (age 1 and 12 months) or apolipoprotein-null (ApoE−/−) (age 1 and 12 months) or green fluorescent protein (GFP)-transgenic mice (limited to adipose tissue harvesting and cell isolation for injections in in vivo experiments, age 12 months) were euthanized. All mice were purchased from The Jackson Laboratory (Sacramento, Calif.). Periepididimal visceral adipose tissue was harvested and adipose-derived mesenchymal stromal cells (AT-MSCs) were isolated by using a modified version of the protocol originally described by Zuk and colleagues²⁴. Parallel experiments were done using bone marrow mesenchymal stromal cells (BM-MSCs) and human mesenchymal stem cells (purchased from from Lonza, Atlanta, Ga.).

Characterization Of Murine Adipose Tissue-Derived Mesenchymal Stromal Cells By Flow Cytometry. For this, AT-MSCs (passage P3, harvested from male C57 mice, age 12 months) were washed with phosphate buffered saline (PBS) and detached by scraping in 3 mmol/L ethylene diamino tetra-acetic acid (EDTA)/Hank's-buffered saline solution (HBSS) without trypsin, as previously described²⁵.

Cloning Of TERT And Myocardin In Lentiviral Expression Plasmids And Lentiviral Production. Full-length cDNAs for human TERT (3.6 kb, Genebank accession number NM_198253.2) and human MYOCD (3.1 kb Genebank accession number NM_153604.1) were amplified by PCR, subcloned and expressed into the pLenti-TOPO cloning vector (Invitrogen, Grant Island, N.Y.). For lentiviral production, all cell culture procedures were performed under biosafety level 2 conditions, accordingly to experimental procedures previously described²⁶.

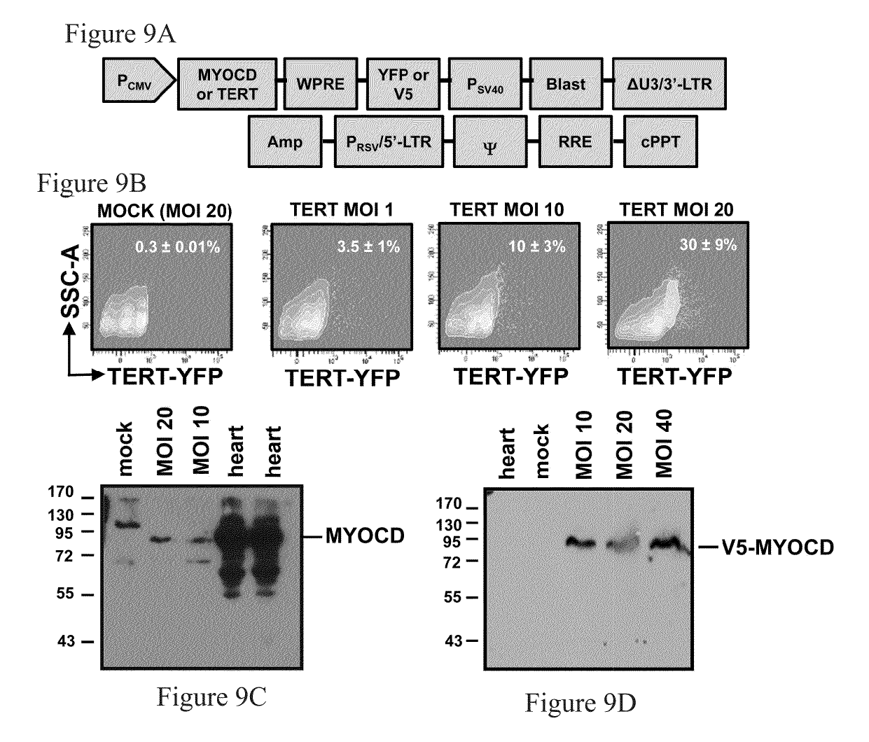

Fluorescence-Activated Cell Sorting (FACS) And Western Analysis Of Transduced Cells. 1×10⁶ murine AT-MSCs or murine BM-MSCs or hMSCs were plated in 60 mm culture dish. Serial dilutions of concentrated lentiviral supernatants were incubated with cells for 16 h in a volume of 10 mL, in the presence of polybrene at 16 pg/mL. Cells were maintained in culture for 5 days, trypsinized, and an aliquot of each preparation was analyzed for TERT-YFP expression by FACSCalibur flow cytometer (BD Biosciences), and for MYOCD-V5 expression by western analysis (FIG. 9B, and FIG. 9C). At 5 days post transduction, an increase in the expression of MYOCD or MYOCD-V5 (FIG. 9C), or in the percentage of cells positive for TERT-YFP (FIG. 9B), correlated with the increase of MOI.

Proliferation And Colony Forming Unit (CFU) Assays. 1×10³/cm² wild-type or lentivirus-transduced murine AT-MSCs were plated in 96-well plate and counted after 1 to 5 days. At each time point, population doubling time (PTD) was calculated using the following equation: (log¹⁰ [N/N0]×3.33), where N is the total number of cells and N0 is the number of seeded cells²⁷. For the Colony Formit Unit (CFU) assay, methylcellulose cultures were performed with wild-type or lentivirus-transduced AT-MSCs, which were trypsinized once and then introduced into the methylcellulose medium (MethoCult MG3534, StemCell Technologies, Vancouver, BC, Canada), all at 1.5×10³ cells/cm² by single-cell plating. Plates were examined under phase-contrast microscopy, and colonies were scored after 14 days from triplicate cultures.

Bromodeoxyuridine Cell Proliferation Assay. Cell proliferation using BrdU (bromodeoxyuridine) incorporation assay was quantified in murine wild-type AT-MSCs, in mock-transduced AT-MSCs, as well as in AT-MSCs transduced with TERT and/or MYOCD. Cells (2×105 cells/mL) at passage 3 were plated in 96 well plates and media were replaced with serum-free media containing 10 μM BrdU (Calbiochem La Jolla, Calif.) at 24 h before harvesting cells. Cells were trypsinized, washed with PBS and fixed in 1% paraformaldehyde in PBS for 15 min, followed by incubation in PBS containing 0.2% Tween-20 for 30 min at 37° C. Cells were then incubated with mouse monoclonal anti-BrdU antibody (Calbiochem) overnight at 4° C., then washed twice, incubated with peroxidase goat anti-mouse secondary antibody (Vector Laboratories, Burlingame, Calif.) for 1 h at room temperature. After 3 washes with PBS, cells were subjected to stop solution, and the absorbance in each well measured using a spectrophotometric plate reader at dual wavelengths of 450-595.

Cytotoxicity Tests And Annexin V/propidium Iodide Staining. A live/dead viability/cytotoxicity kit containing SYTOX Green (Invitrogen, Carlsbad, Calif.) was used to measure the cytotoxicity of lentiviral transduction protocol. Aliquots of 100 μl of murine wild-type or lentivirus-transduced BM-MSCs (mock or TERT/MYOCD transduced cells) at 1×10⁶ cells/mL in PBS were added to 50 μl of 0.5 μM SYTOX Green and incubated for 10 min at room temperature. After washing the percentages of SYTOX Green positive cells (corresponding to dead cells) were determined by flow cytometry using a FACS Vantage instrument (Becton Dickinson Immunocytochemistry Systems, San Jose, Calif.) equipped with 488 nm argon-ion laser. Green fluorescence emission was collected with an emission peak of 523 nm.

For apoptosis assays, 1×10⁶ murine wild-type or lentivirus-transduced BM-MSCs (mock or TERT/MYOCD transduced cells) were plated in 60 mm culture dish and treated overnight with Fas/CD95 (500 ng/mL). Apoptosis was assessed by flow cytometry, by using the annexin V-fluorescein isothiocyanate kit from Pharmingen (Franklin Lakes, N.J.). Cells were washed with cold PBS and resuspended with binding buffer (10 mM HEPES/NaOH, pH 7.4, 140 mM NaCl, 2.5 mmol/L CaCl₂). Annexin V and propidium iodide were added to the cell preparations and incubated for 25 min in the dark. A 1× Binding Buffer (400 μL) [from 10x Binding Buffer with the following composition: 0.1 M HEPES (pH 7.4), 1.4 M NaCl, 25 mM CaCl₂, not provided with the kit] was then added to each tube, and the samples were analyzed by flow cytometry.

Osteogenic and adipogenic differentiation assays. For osteogenic differentiation, cells were plated on 6-well plates at 5×10³ cells/cm². Regular medium was replaced by osteogenic differentiation medium (Stem Pro osteogenesis differentiation kit, Invitrogen) and after 21 days cells were fixed for 30 min in 4% paraformaldehyde and stained with 2% Alizarin Red S solution (3 min) (Sigma). For mineralization quantification, Alizarin Red S precipitate was extracted using a 10% acetic acid/20% methanol solution for 45 minutes. The extracted stain was then transferred to a 96-well plate, and the absorbance at 450 nm was measured using a SpectraMax 340 plate reader/spectrophotometer (Molecular Devices Corp.).

For adipogenic differentiation, cells were plated on 6-well plates at 1×104 cells/cm². Medium was replaced with adipogenic differentiation medium (StemPro adipogenesis differentiation kit, Invitrogen), and after 14 days Oil Red O lipid staining was performed. Cells were washed with PBS, fixed in a 10% solution of formaldehyde (Sigma) for 1 h, washed with 60% isopropanol (Sigma), and stained with an Oil Red O solution (stock solution from Sigma, diluted in 60% isopropanol) for 10 min. Cells were washed with tap water and destained in 100% isopropanol for 15 min. Images were collected using an Olympus (Tokyo, Japan) microscope. For each condition, 2 differentiation and 2 control experiments (nondifferentiation medium) were conducted for each of cell types (murine wild-type AT-MSCs, mock-transduced AT-MSCs, as well as AT-MSCs transduced with TERT and/or MYOCD). Stained oil droplets were dissolved in isopropanol and quantified at 490 nm using a spectrophotometer.

Unilateral Hind Limb Ischemia. 12 weeks-old male ApoE−/− mice (25-30 g) had unilateral hind limb ischemia by ligation of the proximal left femoral artery and vein, using the contralateral limb as a control. One day after femoral ligation, mice (n=5 for each group) were randomly treated by multiple intramuscular (i.m.) injections (3 injections in the adductor, and 5 in the semimembranous muscles) in the ischemic leg, with single dose of: (a) allogeneic mock-transduced MSCs (3×10⁶ cells/500 μl); or (b) allogeneic MSCs transduced with TERT and/or MYOCD (3×10⁶ cells/500 μl); or (c) PBS (500 μl), as a non-cellular control. Blood flow was measured in anesthetized animals one day before ligation (baseline), one day after ligation, and 21 days after injections by using Laser Doppler Perfusion Imager (LDPI) System (PIM II, Perimed).

Immunofluorescence studies and cell engraftment rate determination. Cryosections (5 μm) of hindlimb (treated with PBS, or 3×10⁶ mock-transduced GFP+ MSCs or 3×10⁶ GFP+ MSCs transduced with TERT and MYOCD for 21 days; n=5, 20 sections/leg) were incubated with anti-α-smooth muscle actin (ASMA) antibody (Sigma) overnight at 4° C. Sections were then incubated with phycoerythrin (PE)-conjugated anti-rabbit IgG (Invitrogen, Carlsbad, Calif.). Nuclei were counterstained with DAPI. Immunostained tissues were visualized with a Cambridge Research & Instrumentation (CRi) Nuance multispectral imaging system (Cambridge Research & Instrumentation, Inc., Woburn, Mass., USA)²⁸. A spectral cube for cells, which contains the complete spectral information at 10-nm wavelength intervals from 520 to 720 nm were collected. The resulting images were unmixed using the Nuance system to obtain three images, each corresponding to one of the fluorochromes (GFP, bisbenzimide and PE). Evaluations of the number and distribution of mock-transduced MSCs and MSCs transduced with TERT and MYOCD in the transplanted tissues at postoperative days 21, were performed by counting GFP/bisbenzimide-positive cells. PE positive cells indicated the expression of anti-α-smooth muscle actin (ASMA).

Histological Evaluation Of Capillary And Arteriole Density. The effects of injections of cell suspensions or saline on total vessel density of microcirculation (capillaries and arterioles) was assessed in the 5 μm-thick paraffin sections taken from the adductor and semimembranous muscles from both the ischemic and non-ischemic limbs at postoperative day 21 by immunohistochemistry with anti-von Willebrand factor (vWF) and α-smooth muscle actin (ASMA) antibodies (Sigma). Each hindlimb was transversely cut into 5 equal sections (proximal to distal) and embedded in 5 separate paraffin blocks. Paraffin-embedded limb sections of ApoE−/− mice (12 months old; n=5) were deparaffinized, rehydrated. Sections were stained with a monoclonal antibody directed against ASMA (1:50 dilution) or a monoclonal antibody against vWF (1:100 dilution). Capillaries were identified as vessels that stained positive for vWF, while arterioles were identified as vessels that stained positive for ASMA. Sections were counterstained with hematoxylin to identify nuclei. Arterioles and capillaries were counted in a blinded manner in 5 randomly selected high-power fields at 10× magnification on transverse sections from each hindlimb. Vascular images were taken with the use of an inverted light microscope (Olympus IX71) and analyzed with Image-Pro Plus software (Media Cybernetics). Vessel densities were expressed as the number of arterioles per square millimeter.

Immunoblotting. Total proteins from wild-type or lentivirus-transduced murine BM-MSCs and AT-MSCs, or from ischemic and non-ischemic skeletal muscle tissues of injected mice were isolated in ice-cold RadioImmuno precipitation Assay (RIPA) buffer (Sigma Aldrich). Proteins were separated under reducing conditions and electroblotted onto polyvinylidene fluoride membranes (Immobilon-P; Millipore, Bedford, Mass.). After blocking, the membranes were incubated overnight at 4° C. with the following primary antibodies: (1) Myocardin (R&D Systems), (2) Annexin V (BD Biociences), (3) cardiac actin (Sigma Aldrich), (4) smooth muscle-a actin (Sigma Aldrich), (5) V5-epitope (Invitrogen). Equal loading/equal protein transfers were verified by stripping and reprobing each blot with an anti-beta-actin or an anti-GAPDH antibody (Sigma).

Cloning Of TERT And McA Expression Plasmids And Contructs For The BRET Assay. Full-length cDNAs for murine TERT (3.6 kb) and murine McA (3.1 kb) were amplified by PCR, subcloned into the TOPO cloning vector pcDNA3.1 D/V5-His (Invitrogen) and expressed into the expression vectors pRluc-C3 and pAcGFP1-C2, respectively (Perkin Elmer BioSignal Packard Inc., BD Science Clontech). Detailed methods are reported in the Online Supplemental Material. Murine MSCs were transfected with plasmid DNA (Rluc-McA, pAcGFP-TERT, or empty vector) in the presence of lipofectamine (Invitrogen). Rluc-McA gene expression in MSCs was detected by luciferase activity, while pAcGFP-TERT was detected by the green fluorescence protein. Detailed methods are reported in the Online Supplemental Material.

siRNA-Targeted Silencing Of TERT and MYOCD. A pool of 3 different small interfering RNA (siRNA) oligonucleotides against TERT and McA, scrambled negative control siRNA, and GAPDH positive control siRNA were obtained from Ambion. Briefly, 2×10⁵ cells/well were plated in 6-well plates in low-serum medium without antibiotics (OptiMem, Invitrogen). Cells were incubated with 6 μL of siRNA transfection reagent containing 15 nmol/L of 1 of the following: a mixture of 3 different siRNAs against either TERT or McA; siRNA against GAPDH (positive control); or scrambled siRNA (negative control). Transfection medium was added up to a total volume of 800 μL. After 24 h, fresh medium was added and cells were incubated for an additional 16 h. Cells were harvested by using EDTA/PBS without trypsin, and nuclear proteins were extracted for Western blot analysis with antibodies specific for TERT, McA, and β-actin.

BRET Assay. Bioluminescence resonance energy transfer (BRET) assays were performed as described in the Online Supplement Material. In brief, MSCs transfected with plasmids encoding for Rluc-McA and pAcGFP-TERT, were plated in 96-well plates. The cell permeant luciferase substrate coelenterazine-H (PerkinElmer Life Sciences) was added at a final concentration of 5 μM, and readings with the POLARstar Optima plate reader (BMG Labtechnologies, Offenburg, Germany). For each well, the BRET ratio was calculated as: (E₅₁₅−background₅₁₅)/(E₄₁₀−background₄₁₀), and reported as mBRET (10³×BRET ratio). For BRET saturation curve experiments, cells were transfected with a constant amount of plasmid with the Rluc-McA (4 μg DNA) construct and increasing amounts of plasmid with the pAcGFP-TERT construct (1-4-10 μg DNA). The expression levels of the Rluc- and pAcGFP-tagged constructs in cells transfected with different ratios of plasmids were monitored by separate measurements of total luminescence and total fluorescence on aliquots of transfected cell samples. The calculated BRET signals were plotted as a function of the total fluorescence (RFU)/luminescence (RLU) ratios, and data was analyzed using linear and non-linear regression curve fitting in GraphPad Prism (GraphPad Software, CA, USA).

Telomeric Repeat Amplification Protocol (TRAP) Assays. Telomerase activity was quantified in Dil-acLDL⁺ MSCs and Dil-acLDL⁻ MSCs, as well as in murine embryonic stem cells (ESCs; ATCC) by using a TRAPeze Telomerase Detection Kit (Intergen Chemicon Temecula, Calif.), according to the manufacturer's protocol. Telomere extension was performed at 30° C. for 30 min, followed by 36 cycles of a 3-step PCR (94° C. for 30 sec, 59° C. for 30 sec, and 72° C. for 1 min) and final extension at 72° C. for 3 min. A standard curve of telomerase activity was generated with TSR8 control templates at different concentrations. In all of the PCR reactions, a 36-base pairs template was included as the internal control. In addition, nuclear proteins from HeLa cells were used as a positive control for telomerase activity, and Chaps buffer was used as a negative control for the presence of primer-dimer PCR artifacts and PCR contamination carried over from other samples. The TRAP products were analyzed by electrophoresis in a nondenaturing 12% polyacrylamide gel at 5 V/cm. After electrophoresis, the gels were stained with SYBR green and photographed. TERT activity was measured by reading the fluorescence at excitation/emission settings of 495/516 nm for fluorescein or FITC and 600/620 nm for sulforhodamine.

Statistical Analysis. Data were expressed as mean±standard deviation (SD).Two-group comparisons were performed with the use of a Student t-test for unpaired values. Multiple-group comparisons were made with analysis of variance (ANOVA), with the Mann-Whitney post hoc test was used to determine statistical significance within and between groups (GraphPad Prism 5). A P-value less than 0.05 was considered significant.

Results.

Characterization Of Murine Adipose-Derived Mesenchymal Stromal Cells For Mesenchymal Stem Cell Markers. At flow cytometry, a considerable number of AT-MSCs in primary cultures (at passage P3) displayed in one embodiment endothelial progenitor antigen expression (CD45-CD34+CD133−: 37±24%; CD45-CD133+CD34−: 2±3%; CD45−CD34+CD133+: 6±3%), concomitant with the uptake for DiI-acLDL, mesenchymal stem cell markers CD105, CD44, CD29, CD71, CD106 (4.8±0.2%, 60±4%, 1.4±0.5%, 0.2±0.01%, 0.01±0.00%, respectively) and markers for pericytes and smooth muscle cell lineages (smooth muscle cell α-actin: 47.9±4%; desmin: and 7.4±5.0%, respectively). At higher passages in culture (P>3), a small fraction of adherent cells displayed endothelial progenitor antigen expression or endothelial cell markers (CD31), while the majority of adherent cells were positive for mesenchymal stem cells markers²⁵.

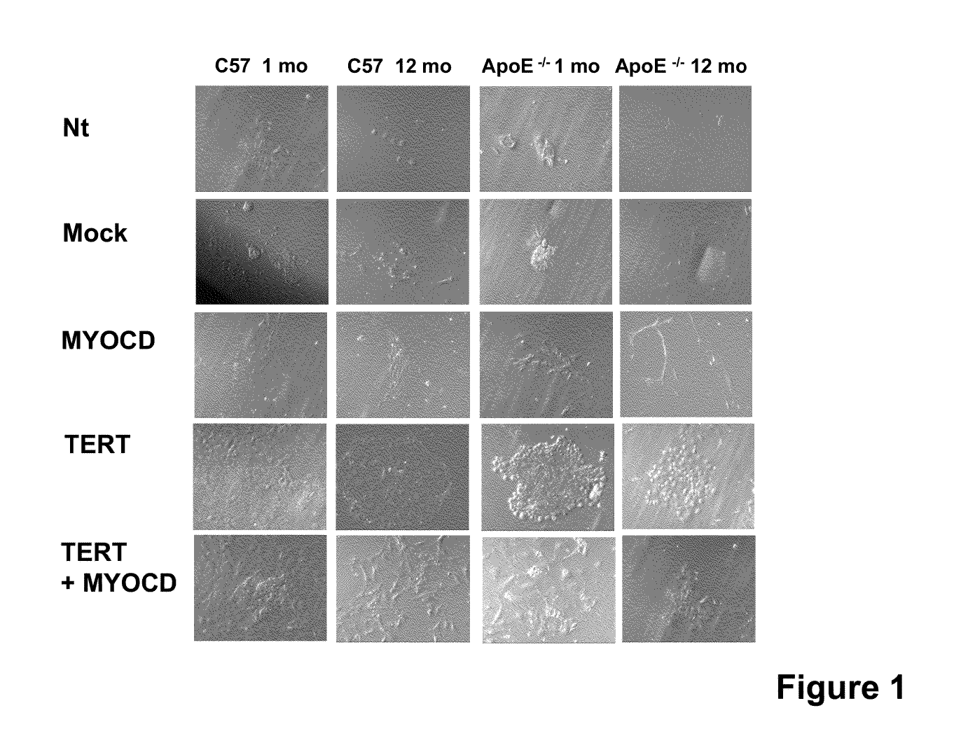

Clonogenic assessment of MSCs overexpressing TERT and MYOCD. In one embodiment the ex vivo proliferative potential of MSCs from C57/BL6 or ApoE−/− overexpressing TERT and MYOCD was assessed by performing in vitro clonogenic assays in methylcellulose, in this assay, individual colonies were theoretically derived from single MSCs and the size of the colonies at a given time reflect their proliferative capacity; all of the colonies from aged mice (C57 and ApoE−/−) were remarkably decreased in number and size compared to young mice (N=5, ° p<0.05 versus aged mice) (FIG. 1 and Table 1). In a further embodiment, TERT-overexpressing MSCs, isolated from the adipose tissue of young (1 month old) and aged (12 months old) mice (C57 or ApoE−/−), formed significantly more and larger colonies than mock-transduced controls, thus indicating that TERT increases the ex vivo proliferative capacity of MSCs (N=5, *p<0.05 versus mock-transduced) (FIG. 1 and Table 1). MYOCD overexpression alone did not inhibit the growth of MSCs. MYOCD did not interfere with TERT-mediated clonogenic activity, but slightly increased the number of MSC clones obtained.

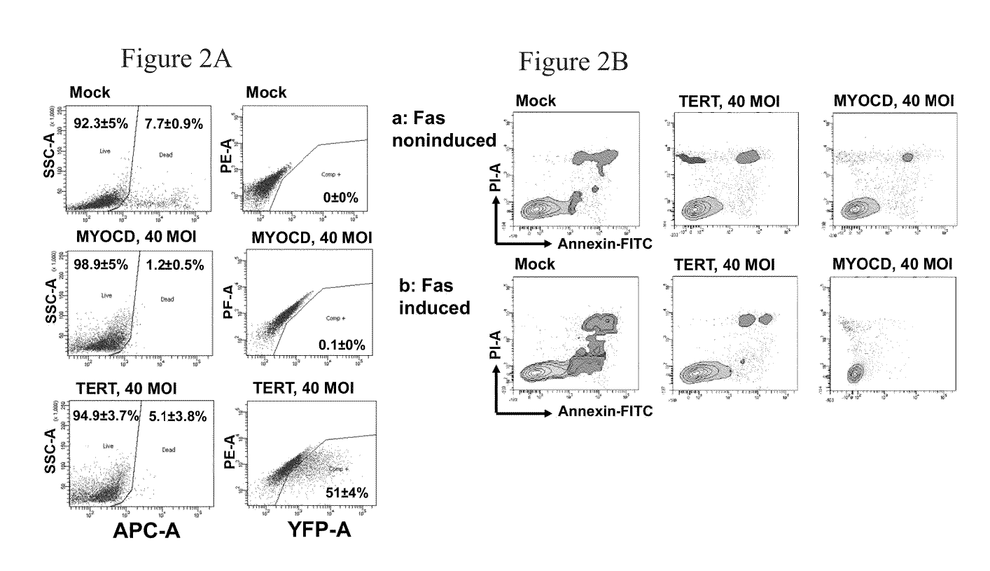

Effect of TERT and MYOCD overexpression on MSC proliferation. In one embodiment, the colonies of TERT-overexpressing cells were much larger than those of the mock-transduced cells (FIG. 1), and therefore further analyzed the impact of TERT and MYOCD overexpression on the growth properties of MSCs. Analysis of cumulative cell numbers over several days indicated that TERT-overexpressing cells had a growth advantage, which was obvious after 3 days in culture, whereas MYOCD overexpression did not inhibit the growth of MSCs. To elucidate the cause of the observed differences, it was investigated whether there were differences in the proliferation or basal cell death rates of these cells. Cultures of cells were labeled with BrdU to monitor proliferating cells and analyzed by spectrophotometry after staining with anti-BrdU antibody. The rates of BrdU incorporation in MSCs from aged mice (C57 and ApoE−/−) were significantly lower compared to young mice (N=5, ° p<0.05 versus aged mice) (Table 2). The number of BrdU-labeled cells in TERT-expressing populations was markedly increased compared with mock-transduced cells (N=5, *p<0.05 versus mock-transduced) (Table 2). MYOCD overexpression alone slightly increased proliferation of MSCs but did not interfere with TERT-mediated proliferative effects. In one embodiment, the effect of TERT and MYOCD overexpression on MSC death was studied by Sytox green fluorescence, which stains only dead/dying cells, both qualitatively and quantitatively (FIG. 2 panel A). An increase in the number of Sytox-positive green cells indicates an increase in cell death because Sytox green dye permeates compromised cell membranes to stain nuclear chromatin.

In one embodiment, quantitative analysis of Sytox fluorescence using flow cytometry revealed a decrease in MSC cell death by MYOCD at day 21 after transduction (1.2±0.5% versus 7.7±0.9%, N=5, **p<0.01 versus mock-transduced) and, to a lesser extent, by TERT overexpression (5.1±3.8% versus 7.7±0.9%, N=5, *p<0.05 versus mock-transduced) (FIG. 2 panel A). Together, these data strongly suggest that TERT and MYOCD overexpression can prevent cytotoxic cell death.

In another embodiment, the effect of TERT and MYOCD modulation on MSC resistance to apoptosis or necrosis was also evaluated. Annexin V and propidium iodide (PI) labeling were therefore quantified by flow cytometry in Fas ligand stimulated MSCs that had been infected with TERT, MYOCD, or mock-vectors three weeks prior. Overexpression of TERT and MYOCD conferred a greater resistance to Fas-induced and Fas-noninduced apoptosis (N=5, *p<0.05 versus mock-transduced) (annexin V labeling) (FIG. 2B and Table 3). Frequencies of both spontaneous cell death (FIG. 2B, top panels) and Fas-induced apoptosis (FIG. 2B, lower panels) were higher in mock-transduced MSCs than in TERT- or MYOCD-transduced MSCs. Overexpression of TERT and, to a greater extent, MYOCD protected MSCs from spontaneous and Fas-induced apoptosis. Overexpressing TERT and MYOCD led to a decrease in the fraction of annexin V-(early apoptosis) and annexin V/PI-(late apoptosis) positive cells in response to Fas ligand (FIG. 2B, bottom panels and Table 3).

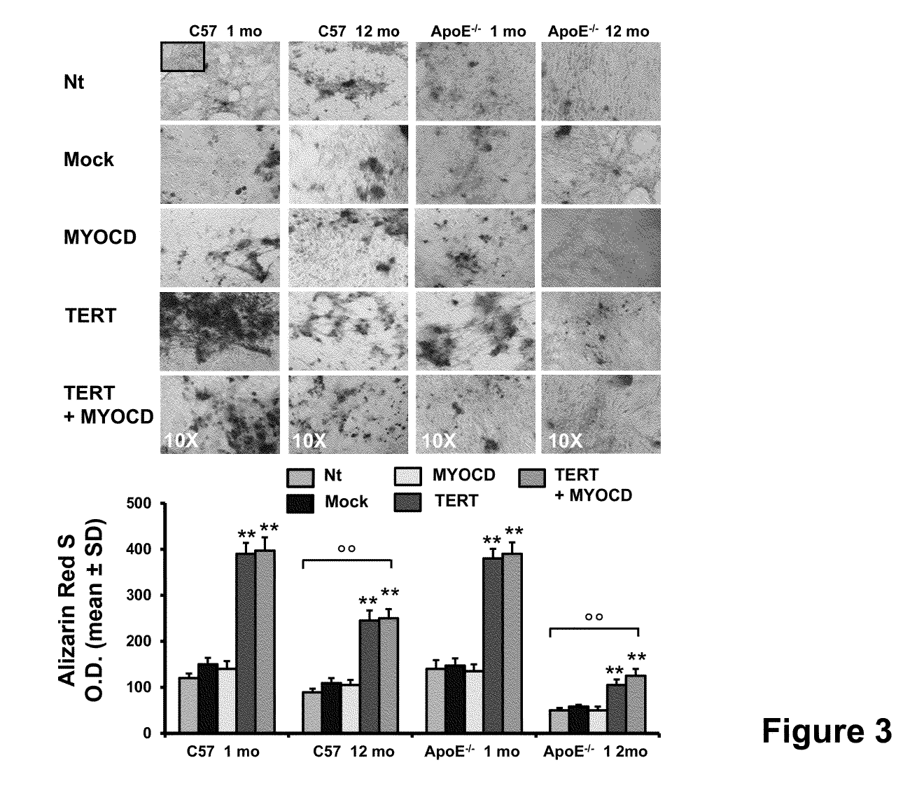

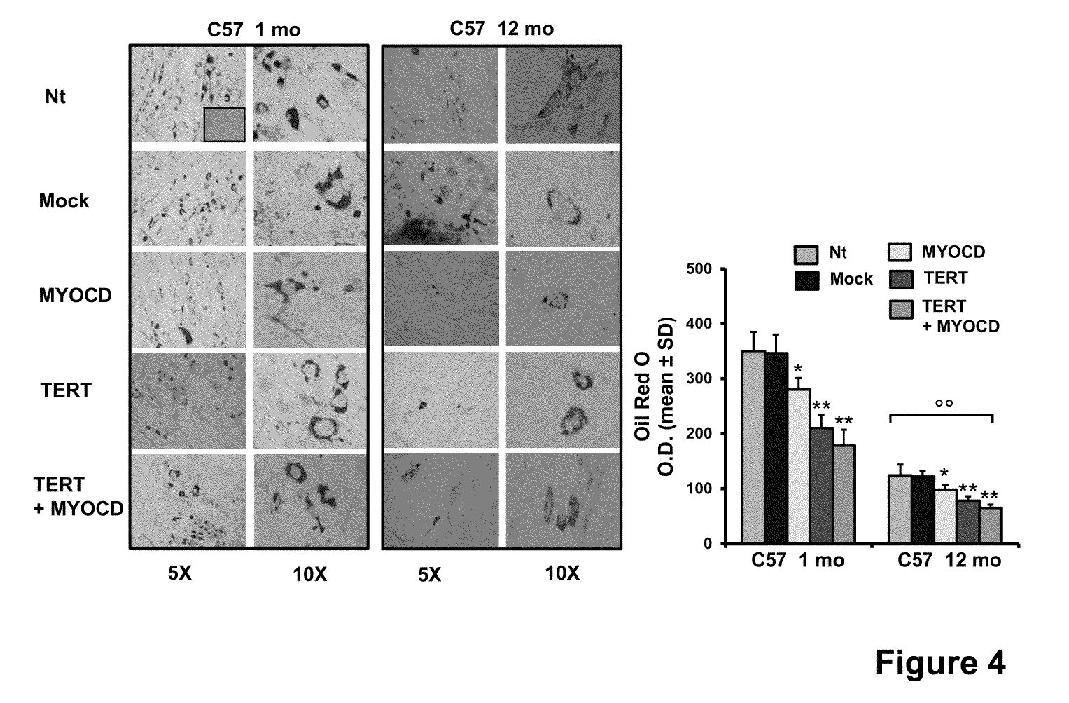

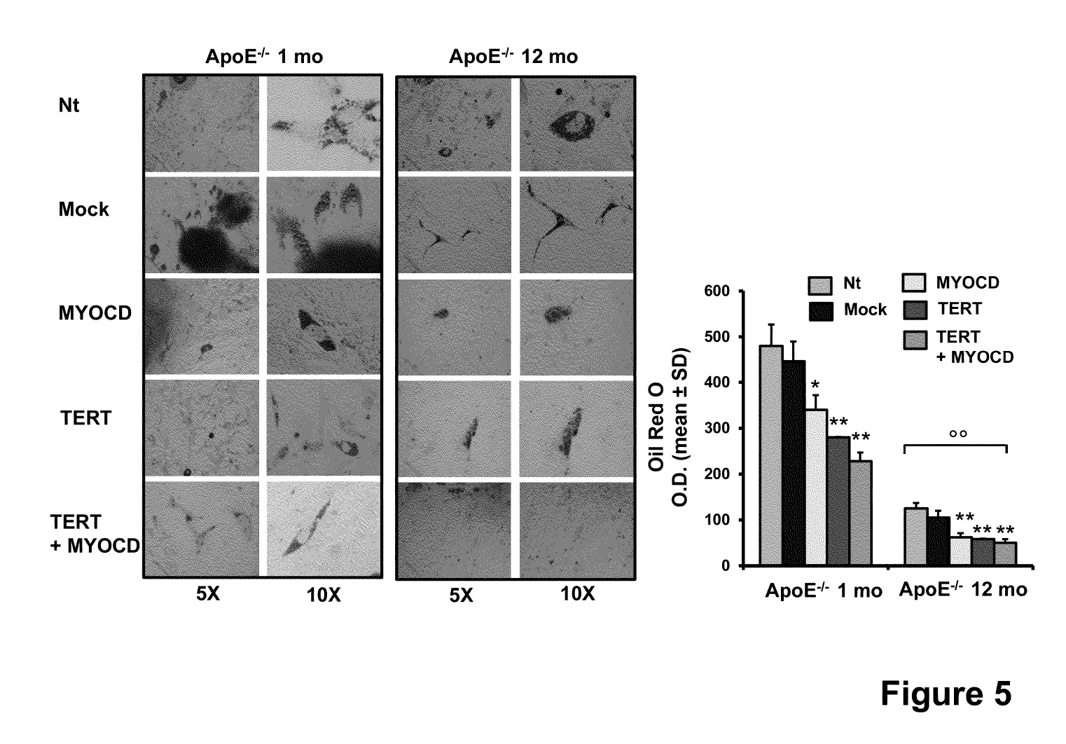

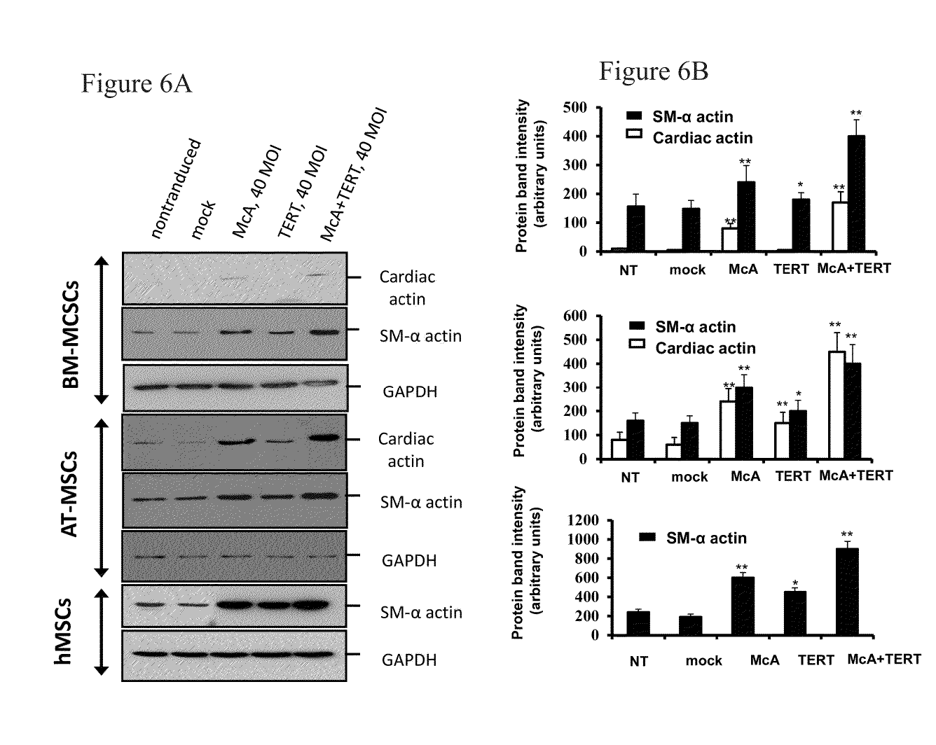

In a further embodiment the effect of TERT and MYOCD overexpression on MSC differentiation was assessed, as it was of interest to determine whether the MSCs overexpressing TERT and MYOCD retained a normal differentiation response. We It was first examined if TERT and MYOCD overexpression altered the normal capabilities of MSCs, such as mesenchymal (osteogenic and adipogenic) and non-mesenchymal (myogenic) differentiation, and directed MSCs toward osteogenic or adipogenic lineages and compared their differentiation efficiency by histochemical staining. It was observed that MSCs from young mice (C57 and ApoE−/−) had higher Alizarin Red S and Oil Red O staining compared with aged mice at day 21 of osteogenic differentiation (N=5, ° ° p<0.01 vs. young mice) (FIG. 3) and day 14 of adipogenic differentiation (N=5, ° ° p<0.01 vs. young mice) (FIGS. 4 and 5), respectively. TERT-transduced MSCs, isolated from adipose tissue of young (1 month old) and aged (12 months old) mice (C57 or ApoE−/−), had an elevated osteogenic differentiation potential compared with mock-transduced MSCs (N=5, **p<0.01 versus mock-transduced) (FIG. 3), whereas they had decreased adipogenic differentiation potential (N=5, **p<0.01 versus mock-transduced) (FIGS. 4 and 5). Thus, overexpression of TERT increased osteogenic potential, and there was an inverse relationship between osteogenic and adipogenic differentiation. MYOCD overexpression did not interfere with TERT-mediated osteogenic differentiation, while it decreased the adipogenic differentiation of MSCs (N=5, *p<0.05 versus mock-transduced) and potentiated the TERT-mediated reduction of adipogenic differentiation (N=5, **p<0.01 versus TERT-transduced) (FIGS. 4 and 5). In a further embodiment myogenic differentiation was compared with mock-transduced MSCs, the cells overexpressing TERT and MYOCD expressed increased myogenic lineage-specific markers (smooth muscle-alpha actin and cardiac actin, N=5, *p<0.05 and **p<0.01 versus mock-transduced) (FIG. 6A-6B). Overall, the results from differentiation assays suggested that the number of progenitor cells derived from TERT- or MYOCD-transduced MSCs is higher because of either faster proliferation and better survival in culture.

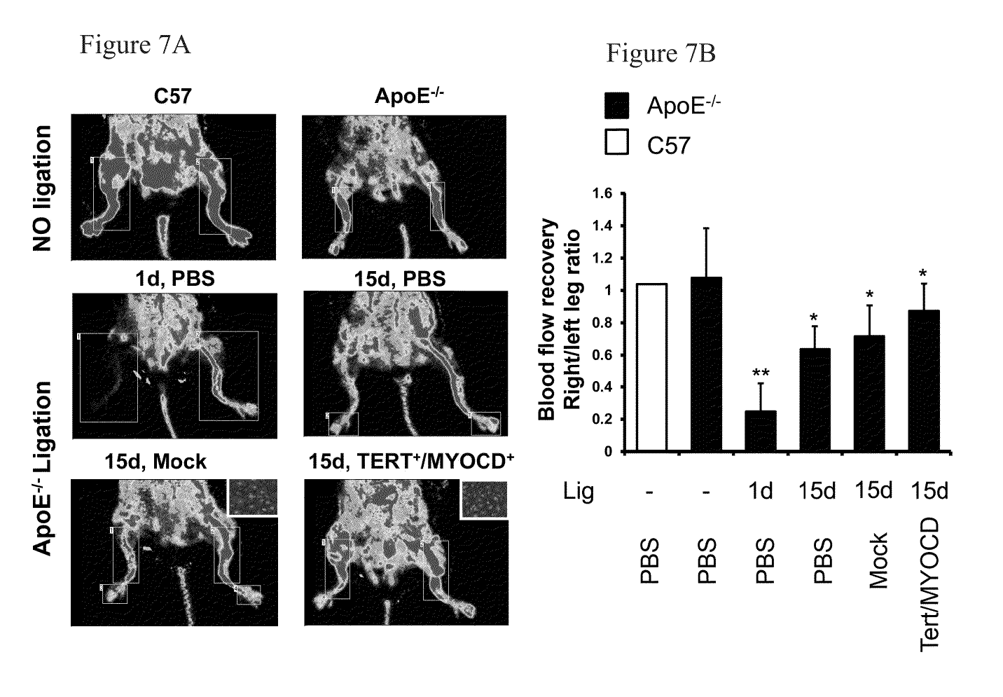

Physiological impact in vivo after transplantation of TERT- and MYOCD-transduced MSCs. In some embodiments, mock- or TERT/MYOCD-transduced MSCs from GFP+ mice were transplanted into the ischemic hindlimbs of ApoE−/− mice, at a single dose of 3×10⁶ cells by multiple intramuscular injections to assess the corresponding physiological impact in vivo after TERT and MYOCD gene transfer. Blood flow was measured before femoral ligation, 1 day after ligation and before treatments, and 2 weeks after treatments. Representative laser Doppler images (FIG. 7A) illustrate perfusion of the ischemic (right) legs versus the non-ischemic contralateral limbs. In nonischemic limbs before femoral ligation, baseline blood flow was better in C57 mice than in ApoE−/− mice. Two weeks after induction of ischemia, mock-transduced GFP+ MSC-treated mice showed a moderate but significantly greater recovery of limb perfusion measured by laser Doppler-derived blood flow compared to mice treated with PBS (N=5, P<0.05) (FIGS. 7A and 7B). Recovery of the ischemic limb was significantly improved among mice transplanted with TERT/MYOCD versus mock-transduced/GFP+ MSCs (N=5, P<0.05) (FIGS. 7A and 7B).

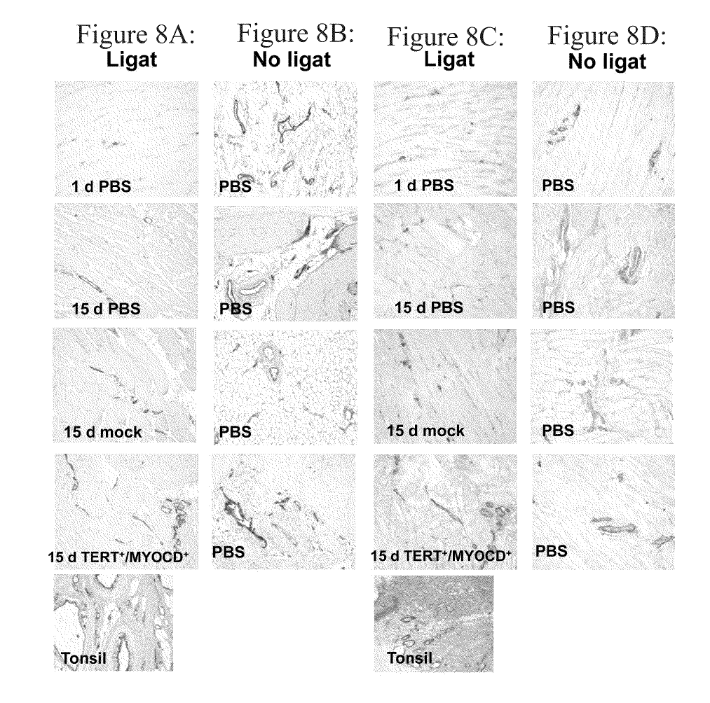

Cell-mediated TERT and MYOCD gene transfer results in significantly greater arteriogenesis in the ischemic legs of ApoE−/− mice. In one embodiment, as neovascularization is believed to be essential for maintaining perfusion recovery, arteriogenesis after cell therapy was studied. To identify arterioles and capillaries, tissue sections of ischemic and contralateral nonischemic legs with anti-α-smooth muscle actin (ASMA) and anti-von Willebrand Factor antibodies respectively were immunostained at 21 days after treatments. Capillary (FIG. 8A and FIG. 8B) and arteriole density (FIG. 8A and FIG. 8D, Table 4) were markedly increased in mice receiving mock-transduced/GFP+MSCs (N=5, P<0.05). Capillary and arteriole density were further improved after transplantation of TERT/MYOCD-transduced/GFP+ MSCs. (N=5, P<0.05). Animals treated with TERT/MYOCD or mock-transduced/GFP+ MSCs showed no evidence of neoplastic transformation.

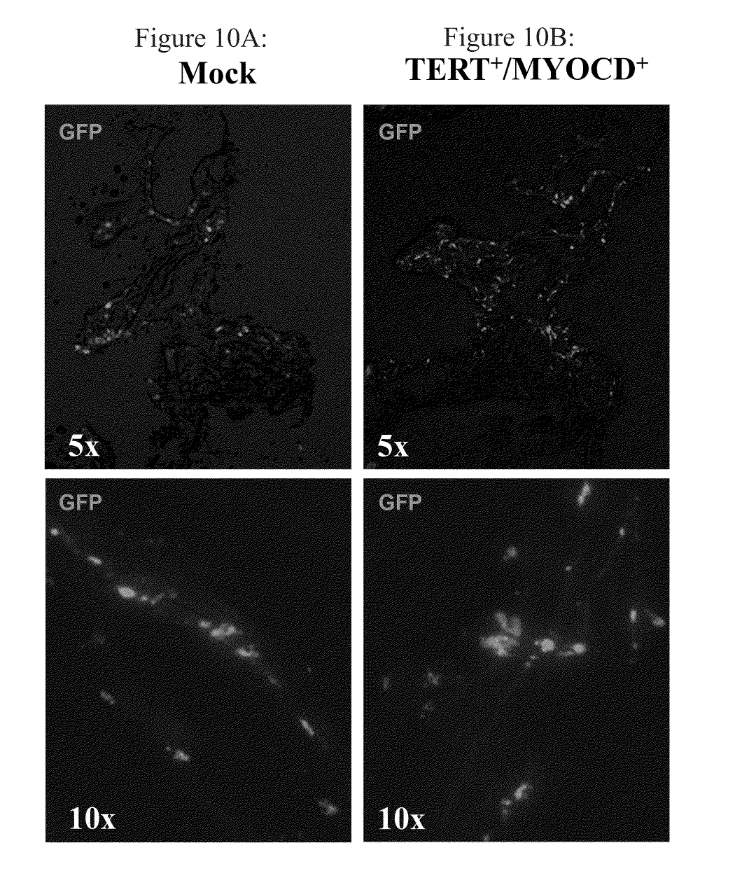

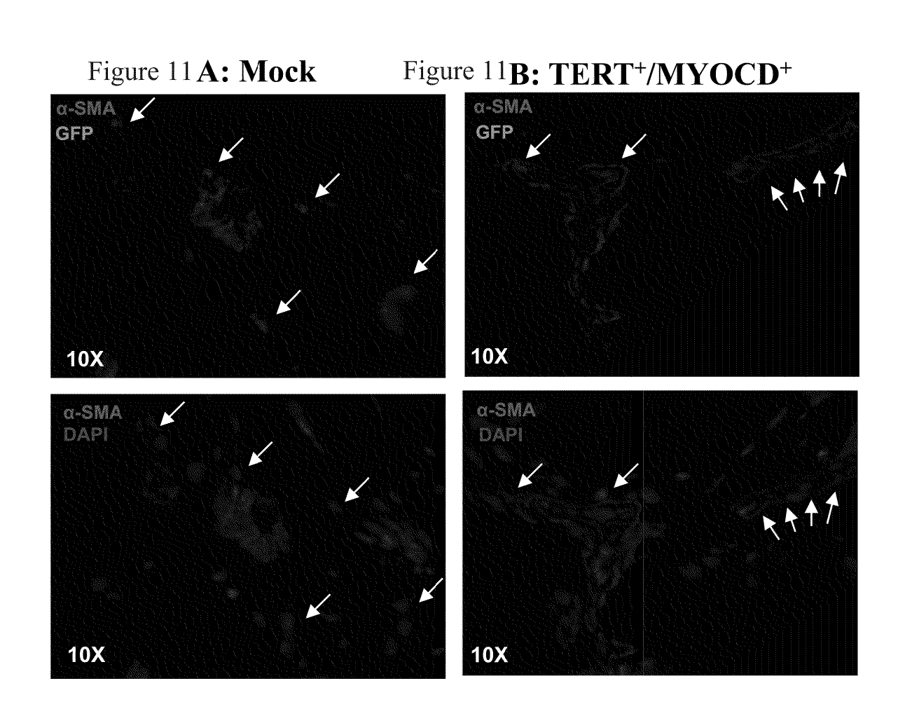

In one embodiment, TERT- and MYOCD-transduced MSCs engraft into ischemic tissue and differentiate into vascular structures. To examine whether TERT/MYOCD and mock-transduced/GFP+ MSCs incorporate into hindlimbs and eventually differentiate into vascular cells a single dose of 3×10⁶ cells was delivered into the ischemic legs of ApoE−/− mice and euthanized the mice 21 days after cell delivery. Long-term engraftment of injected cells was assessed histologically on transverse sections of cell-treated legs. Cell retention rates after 21 days from injections of mock-transduced GFP+ MSCs and TERT/MYOCD-transduced GFP+ MSCs are illustrated in FIG. 10 A and B, respectively. In one embodiment, it was observed that GFP+ cells (green fluorescence) in the skeletal muscles in the areas of MSC injections. No GFP+ cells were found in the skeletal muscles without cell delivery (FIG. 10, panels A and B). Cell retention numbers of mock-transduced GFP+ MSCs at postoperative day 21 were much lower than TERT/MYOCD-transduced GFP+ MSCs, indicating an increase of cell engraftment or proliferation in vivo by TERT and MYOCD overexpression. The quantitation of cell retention is shown in Table 5. Morphometric analysis with the CRi Nuance multispectral imaging system was used to quantify the colocalization of GFP expression with nuclei (DAPI) and smooth muscle signals. It was determined that authentic GFP+ cells incorporated into arterioles of recipient mouse legs (FIG. 11A-11B). Multispectral imaging of transverse leg sections stained for ASMA revealed vascular differentiation of transplanted GFP+ MSCs in ischemic legs at 21 days (n=5). Transplanted GFP+ MSCs gave rise to smooth muscle structure in the muscle tissue of ApoE−/− mice (FIG. 11A-11B). Colocalization of GFP with DAPI and smooth muscle signals (FIG. 11A-11B) was found confirming the differentiation of GFP+ MSCs into smooth muscle cells. Thus, demonstrating that MSCs can integrate into host structures and serve as common vascular progenitors for postnatal arteriogenesis. Colocalization rates of GFP with DAPI and smooth muscle signals in the muscle tissue of ApoE−/− mice transplanted with mock-transduced GFP+ MSCs at postoperative day 21 were much lower than TERT/MYOCD-transduced GFP+ MSCs, indicating an increase in arteriogenesis by TERT and MYOCD overexpression. Although GFP+ MSCs integrated into vascular structures, functional arterioles that originated entirely from GFP+ MSCs were not located, suggesting that vascular direct differentiation of this cell type into newly formed arterioles is a possible, although not necessarily the sole mechanism that mediates arteriogenesis.

Discussion.

The capacity of organs to repair themselves diminishes with age, and maybe due to a reduced functional capability of stem cells²⁹. MSCs in the aged body are susceptible to age-related changes, including high rates of apoptosis and senescence³⁰, and are thus less able to contribute to the endogenous repair process³¹. Furthermore, as the age of the donor increases, the effectiveness of MSC transplantation for age-related diseases diminishes^{32, 33}. In some embodiments described herein, evidence is presented that there may be an interplay between two functionally different nuclear proteins, TERT and MYOCD, in the conversion of aged MSCs to rejuvenating promyogenic stem cells. In some embodiments, delivery of the TERT and MYOCD genes resuscitated MSCs from aged mice by increasing those cells' capacity for survival, proliferation, and myogenic differentiation. In further embodiments, TERT+/MYOCD+ MSCs from adipose tissue improved blood flow and promoted arteriogenesis in ischemic hindlimb. Overexpression of TERT and MYOCD was sufficient to restore regenerative capacity to MSCs derived from old mice. Thus, transplantation of adipose tissue MSCs after rejuvenation in vitro by TERT and MYOCD gene transfer.

It has been demonstrated that the maintenance of telomerase activity during the differentiation of embryonic stem cells (ESCs) enhances proliferation, resistance to apoptosis, and improves differentiation toward hematopoietic lineages by expansion of the progenitor population³⁴. In telomerase-deficient mice experiencing severe tissue degeneration and exhibiting significant progeroid phenotypes, the endogenous telomerase-mediated restoration of telomere function resumed proliferation in quiescent cultures and eliminated degenerative phenotypes across multiple organs including the testes, brain, spleen, and intestines³⁵. In aged MSCs, TERT promotes cell growth and self-renewal by disrupting p53 activity and enhances cell migration through cortactin deacetylation³⁶. Finally, mice with conditional ablation of the MYOCD gene in cardiomyocytes have increased apoptosis and rapid progression of dilated cardiomyopathy and heart failure¹⁹. In agreement with these previous reports, embodiments described herein, indicate that aged MSCs that overexpress TERT and MYOCD have increased proliferation, self-renewal, and differentiation potentials. Similarly, embodiments described herein indicate greater tolerance for apoptosis in aged MSCs mainly after MYOCD and, to a lesser extent, TERT gene transfer, partially in agreement with Tang et al. who have previously shown that myocardin functions as an antiproliferative factor in smooth muscle cells by interfering with NF-kappaB-dependent cell-cycle regulation, without inducing apoptosis³⁷. The process of angiogenesis involves proliferation and survival of transplanted MSCs into the site of ischemic injury followed by myogenic differentiation of these cells. The observed differences in stemness properties and the survival of MSCs related with MYOCD and TERT gene transfer are reflected in their angiogenic potential. The transplantation of MSCs into ischemic tissue assessed the angiogenic potential of aged MSCs after MYOCD and TERT gene expression and showed better capillary and arteriole formation than mock-transduced MSCs. The pro-angiogenic property of TERT+/MYOCD+ MSCs can be explained by their direct regeneration of smooth muscle cells in ischemic muscles following expansion of the myogenic progenitor population.

An animal model that resembles human peripheral artery disease (PAD) is crucial for examining and validating the potential benefits of any novel cell type in a preclinical study. PAD resulting from atherosclerosis produces chronic ischemia. Mice, 8-12 months old, that are deficient in apolipoprotein E (ApoE−/−) and fed normal chow develop spontaneous atherosclerosis that narrows the vessel lumen, which leads to the progressive restriction of blood flow at multiple arterial branches, including hindlimb vessels³⁸⁻⁴⁰. In agreement with these previous reports, in some embodiments of the current invention it has been observed herein that in nonischemic limbs before femoral ligation, baseline blood flow was better in C57 mice than in ApoE−/− mice. Therefore, 12-month-old ApoE−/− mice were used herein as cell therapy recipients because they develop chronic atherosclerosis similar to the atherosclerotic lesions observed in humans.

Further embodiments enforce that TERT may have a role in determining the “myogenic stemness” of MSCs, i.e., maintaining MSCs in an intermediate “biologic window” in which an undifferentiated, uncommitted stem cell evolves toward myogenic commitment while maintaining potency for proliferation¹⁴. Furthermore, embodiments herein reinforce the concept that MYOCD and TERT may synergize in promoting promyogenic gene expression and maintaining the growth capacity of MSCs¹⁶.

In some embodiments, whether TERT-expressing MSCs acquired characteristics of cancer cells, such as anchorage-independent growth in culture or tumorigenicity in mice after transplantation was studied. No such neoplasticity, after TERT transduction by lentiviral constructs was displayed. TERT overexpression by lentiviral transduction was limited to 4 weeks and did not bring about the immortalization of MSCs.

Rodent model often can be translated to humans, hence embodiments herein suggest a novel strategy for the alleviation of stem cell senescence and for enhancing the host response to ischemia in aged patients. Such embodiments further indicate that MSCs transduced with TERT and MYOCD may provide an unlimited source of myogenic cells for therapeutic use in heart and vessel regeneration. This in turn suggests, gene transfer of TERT and MYOCD into MSCs in vitro may offer an option for overcoming the relative paucity of MSCs that can be isolated from adipose tissue in older and sick patients⁴¹. Therefore “rejuvenated” MSCs may be obtained through in vitro modulation of autologous adipose tissue or bone marrow cells even if harvested late in life or after the appearance of organ disease. Although the fundamental mechanisms underlying senescence of mammalian cells (and senescence of the vasculature) remain to be elucidated, findings herein indicate that impairment of the vascular response in aged individuals may be partially restored through transplantation with adipose MSCs following their rejuvenation in vitro by TERT and MYOCD gene transfer.

The concept of “rejuvenating” MSCs via a delay in senescence and enhanced regenerative properties may thus have therapeutic implications for vascular disorders, including myocardial ischemia and PAD and critical limb ischemia (CLI), in which the viability of MSCs and fully differentiated endothelial and smooth muscle cells is recurrently subjected to a variety of individual and environmental stress factors.

In some embodiments of the invention herein described, TERT and MYOCD gene transfer may rejuvenate and restore the myogenic development of aged MSCs from adult adipose tissue. The interaction between TERT and MYOCD in myogenic MSCs may be important in the timing of myogenesis and in the proliferation and differentiation of MSCs. MSCs transduced with TERT and MYOCD may therefore have therapeutic applications for use in the repair and regeneration of ischemic tissues.

### REFERENCES

1. Heeschen C, Lehmann R, Honold J, Assmus B, Aicher A, Walter D H, Martin H, Zeiher A M, Dimmeler S. Profoundly reduced neovascularization capacity of bone marrow mononuclear cells derived from patients with chronic ischemic heart disease. Circulation. 2004; 109(13): 1615-1622.
2. Park J A, Kwon Y G. Could circulating progenitor cell count be a barometer for coronary artery disease progression? Circ J. 2010; 74(9):1804-1805.
3. Werner N, Kosiol S, Schiegl T, Ahlers P, Walenta K, Link A, Bohm M, Nickenig G. Circulating endothelial progenitor cells and cardiovascular outcomes. N Engl J Med. 2005; 353(10): 999-1007.
4. Trensz F, Haroun S, Cloutier A, Richter M V, Grenier G. A muscle resident cell population promotes fibrosis in hindlimb skeletal muscles of mdx mice through the Wnt canonical pathway. Am J Physiol Cell Physiol. 2010; 299(5):C939-947.
5. Brack A S, Conboy M J, Roy S, Lee M, Kuo C J, Keller C, Rando T A. Increased Wnt signaling during aging alters muscle stem cell fate and increases fibrosis. Science. 2007; 317(5839):807-810.
6. Carlson M E, Suetta C, Conboy M J, Aagaard P, Mackey A, Kjaer M, Conboy I. Molecular aging and rejuvenation of human muscle stem cells. EMBO Mol Med. 2009; 1(8-9):381-391.
7. Rauscher F M, Goldschmidt-Clermont P J, Davis B H, Wang T, Gregg D, Ramaswami P, Pippen A M, Annex B H, Dong C, Taylor D A. Aging, progenitor cell exhaustion, and atherosclerosis. Circulation. 2003; 108(4):457-463.
8. Tang X L, Rokosh D G, Guo Y, Bolli R. Cardiac progenitor cells and bone marrow-derived very small embryonic-like stem cells for cardiac repair after myocardial infarction. Circ J. 2010; 74(3):390-404.
9. Hosoda T, Kajstura J, Leri A, Anversa P. Mechanisms of myocardial regeneration. Circ J. 2010; 74(1):13-17.
10. Miranville A, Heeschen C, Sengenes C, Curat C A, Busse R, Bouloumie A. Improvement of postnatal neovascularization by human adipose tissue-derived stem cells. Circulation. 2004; 110(3):349-355.
11. Fraser J K, Schreiber R, Strem B, Zhu M, Alfonso Z, Wulur I, Hedrick M H. Plasticity of human adipose stem cells toward endothelial cells and cardiomyocytes. Nat Clin Pract Cardiovasc Med. 2006; 3 Suppl 1:S33-37.
12. Gaustad K G, Boquest A C, Anderson B E, Gerdes A M, Collas P. Differentiation of human adipose tissue stem cells using extracts of rat cardiomyocytes. Biochem Biophys Res Commun. 2004; 314(2):420-427.
13. Traktuev D O, Merfeld-Clauss S, Li J, Kolonin M, Arap W, Pasqualini R, Johnstone B H, March K L. A population of multipotent CD34-positive adipose stromal cells share pericyte and mesenchymal surface markers, reside in a periendothelial location, and stabilize endothelial networks. Circ Res. 2008; 102(1):77-85.
14. Madonna R, Willerson J T, Geng Y J. Myocardin a enhances telomerase activities in adipose tissue mesenchymal cells and embryonic stem cells undergoing cardiovascular myogenic differentiation. Stem Cells. 2008; 26(1):202-211.
15. Kocher A A, Schuster M D, Szabolcs M J, Takuma S, Burkhoff D, Wang J, Homma S, Edwards N M, Itescu S. Neovascularization of ischemic myocardium by human bone-marrow-derived angioblasts prevents cardiomyocyte apoptosis, reduces remodeling and improves cardiac function. Nat Med. 2001; 7(4):430-436.
16. Madonna R, Wu D, Wassler M, De Caterina R, Willerson J, Geng Y. Myocardin-A Enhances Expression of Promyogenic Genes without Depressing Telomerase Activity in Adipose Tissue-Derived Mesenchymal Stem Cells. Int J Cardiol. 2012; in press.
17. Ueyama T, Kasahara H, Ishiwata T, Nie Q, Izumo S. Myocardin expression is regulated by Nkx2.5, and its function is required for cardiomyogenesis. Mol Cell Biol. 2003; 23(24):9222-9232.
18. Wang Z, Wang D Z, Pipes G C, Olson E N. Myocardin is a master regulator of smooth muscle gene expression. Proc Natl Acad Sci USA. 2003; 100(12):7129-7134.
19. Huang J, Min Lu M, Cheng L, Yuan L J, Zhu X, Stout A L, Chen M, Li J, Parmacek M S. Myocardin is required for cardiomyocyte survival and maintenance of heart function. Proc Natl Acad Sci USA. 2009; 106(44): 18734-18739.
20. Cao X L, Hu X M, Hu J Q, Zheng W X. Myocardin-related transcription factor-A promoting neuronal survival against apoptosis induced by hypoxia/ischemia. Brain Res. 2011; 1385:263-274.
21. Qu Y, Duan Z, Zhao F, Wei D, Zhang J, Tang B, Li J, Yang C, Mu D. Telomerase reverse transcriptase upregulation attenuates astrocyte proliferation and promotes neuronal survival in the hypoxic-ischemic rat brain. Stroke. 2011; 42(12):3542-3550.
22. Jan H M, Wei M F, Peng C L, Lin S J, Lai P S, Shieh M J. The use of polyethylenimine-DNA to topically deliver hTERT to promote hair growth. Gene Ther. 2011; 19(1):86-93.
23. Madonna R, De Caterina R, Willerson J T, Geng Y J. Biologic function and clinical potential of telomerase and associated proteins in cardiovascular tissue repair and regeneration. Eur Heart J. 2011; 32(10):1190-1196.
24. Zuk P A, Zhu M, Mizuno H, Huang J, Futrell J W, Katz A J, Benhaim P, Lorenz H P, Hedrick M H. Multilineage cells from human adipose tissue: Implications for cell-based therapies. Tissue Eng. 2001; 7:211-228.
25. Madonna R, De Caterina R. In vitro neovasculogenic potential of resident adipose tissue precursors. Am J Physiol Cell Physiol. 2008; 295(5):C1271-1280.
26. Madonna R, Bolli R, Rokosh G, De Caterina R. Long-Term Engraftment and Angiogenic Properties of Lentivirally Transduced Adipose Tissue-Derived Stromal Cells. Mol Biotechnol. 2012.
27. Stolzing A, Jones E, McGonagle D, Scutt A. Age-related changes in human bone marrow-derived mesenchymal stem cells: consequences for cell therapies. Mech Ageing Dev. 2008; 129(3): 163-173.
28. http://www.cri-inc.com.
29. Zenovich A G, Taylor D A. Atherosclerosis as a disease of failed endogenous repair. Front Biosci. 2008; 13:3621-3636.
30. Stenderup K, Justesen J, Clausen C, Kassem M. Aging is associated with decreased maximal life span and accelerated senescence of bone marrow stromal cells. Bone. 2003; 33(6): 919-926.
31. Zhang J H, Sampogna S, Morales F R, Chase M H. Age-related ultrastructural changes in hypocretinergic terminals in the brainstem and spinal cord of cats. Neurosci Left. 2005; 373(3):171-174.
32. Perin E C, Silva G V, Zheng Y, Gahremanpour A, Canales J, Patel D, Fernandes M R, Keller L H, Quan X, Coulter S A, Moore W H, Herlihy J P, Willerson J T. Randomized, double-blind pilot study of transendocardial injection of autologous aldehyde dehydrogenase-bright stem cells in patients with ischemic heart failure. Am Heart J. 2012; 163(3):415-421, 421 e411.
33. Perin E C, Willerson J T, Pepine C J, Henry T D, Ellis S G, Zhao D X, Silva G V, Lai D, Thomas J D, Kronenberg M W, Martin A D, Anderson R D, Traverse J H, Penn M S, Anwaruddin S, Hatzopoulos A K, Gee A P, Taylor D A, Cogle C R, Smith D, Westbrook L, Chen J, Handberg E, Olson R E, Geither C, Bowman S, Francescon J, Baraniuk S, Piller L B, Simpson L M, Loghin C, Aguilar D, Richman S, Zierold C, Bettencourt J, Sayre S L, Vojvodic R W, Skarlatos S I, Gordon D J, Ebert R F, Kwak M, Moye L A, Simari R D. Effect of transendocardial delivery of autologous bone marrow mononuclear cells on functional capacity, left ventricular function, and perfusion in chronic heart failure: the FOCUS-CCTRN trial. JAMA. 2012; 307(16):1717-1726.
34. Armstrong L, Saretzki G, Peters H, Wappler I, Evans J, Hole N, von Zglinicki T, Lako M. Overexpression of telomerase confers growth advantage, stress resistance, and enhanced differentiation of ESCs toward the hematopoietic lineage. Stem Cells. 2005; 23(4):516-529.
35. Jaskelioff M, Muller F L, Paik J H, Thomas E, Jiang S, Adams A C, Sahin E, Kost-Alimova M, Protopopov A, Cadinanos J, Homer J W, Maratos-Flier E, Depinho R A. Telomerase reactivation reverses tissue degeneration in aged telomerase-deficient mice. Nature. 2011; 469(7328):102-106.
36. Zhang Y, Zhang M, Dong H, Yong S, Li X, Olashaw N, Kruk P A, Cheng J Q, Bai W, Chen J, Nicosia S V, Zhang X. Deacetylation of cortactin by SIRT1 promotes cell migration. Oncogene. 2009; 28(3):445-460.
37. Tang R H, Zheng X L, Callis T E, Stansfield W E, He J, Baldwin A S, Wang D Z, Selzman C H. Myocardin inhibits cellular proliferation by inhibiting NF-kappaB(p65)-dependent cell cycle progression. Proc Natl Acad Sci USA. 2008; 105(9):3362-3367.
38. Reddick R L, Zhang S H, Maeda N. Atherosclerosis in mice lacking apo E. Evaluation of lesional development and progression. Arterioscler Thromb. 1994; 14(1):141-147.
39. Meir K S, Leitersdorf E. Atherosclerosis in the apolipoprotein-E-deficient mouse: a decade of progress. Arterioscler Thromb Vasc Biol. 2004; 24(6):1006-1014.
40. Wouters K, Shiri-Sverdlov R, van Gorp P J, van Bilsen M, Hofker M H. Understanding hyperlipidemia and atherosclerosis: lessons from genetically modified apoe and IdIr mice. Clin Chem Lab Med. 2005; 43(5):470-479.
41. Madonna R, Renna F V, Cellini C, Cotellese R, Picardi N, Francomano F, Innocenti P, De Caterina R. Age-dependent impairment of number and angiogenic potential of adipose tissue-derived progenitor cells. Eur J Clin Invest. 2011; 41(2):126-133.

The above discussion is meant to be illustrative of the principles and various embodiments of the present invention. Numerous variations and modifications will become apparent to those skilled in the art once the above disclosure is fully appreciated. It is intended that the following claims be interpreted to embrace all such variations and modifications.

TABLE 1
The effect of TERT and MYOCD transduction on CFU formation
of murine AT-MSCs from young and aged C57 and ApoE^{-/-} mice

| | Nt | Mock | MYOCD | TERT | MYOCD⁺TERT⁺ |
|---|---|---|---|---|---|
| C57 1 mo | 5 ± 2 | 4 ± 1 | 5 ± 3 | 11 ± 5 * | 13 ± 6* |
| C57 12 mo | 1 ± 2° | 1 ± 1° | 2 ± 1° | 6 ± 2*° | 8 ± 2*° |
| apoE ^{-/-} 1 mo | 6 ± 2 | 5 ± 1 | 6 ± 4 | 12 ± 4* | 15 ± 7* |
| apoE^{-/-} 12 mo | 1 ± 1° | 1 ± 1° | 2 ± 1° | 4 ± 2*° | 5 ± 2*° |

Values are mean ± SD of counted CFU/10⁶ cells. Periepidydimal adipose tissue was harvested from C57 or ApoE^{-/-} mice, 1 month and 12 months old. Adipose tissue-derived mesenchymal stromal cells (AT-MSCs) were isolated and plated in methylcellulose at passage 3. Colonies were counted after 2 week from low-density plating. The number of CFU colonies was determined by counting 8 different high-power fields (h.p.f) using a 10 × objective. Fields for counting CFU were randomly located at half-radius distance from the center of the monolayers.
N = 5 mice/group;
°p < 0.05, versus age 1 mo;
*p < 0.05, versus mock-transduced.
Legend:
Nt, nontransduced,
MYOCD, transduced with pLenti-myocardin vector,
TERT, transduced with pLenti-TERT vector.

TABLE 2
The effect of TERT and MYOCD transduction on cell proliferation
of murine AT-MSCs from young and aged C57 and ApoE^{-/-} mice

| | Nt | Mock | MYOCD | TERT | MYOCD + TERT |
|---|---|---|---|---|---|
| C57 1 mo | 0.342 ± 0.02 | 0.353 ± 0.03 | 0.339 ± 0.03 | 0.465 ± 0.09* | 0.450 ± 0.03* |
| C57 12 mo | 0.154 ± 0.05° | 0.134 ± 0.05° | 0.129 ± 0.01° | 0.224 ± 0.05*° | 0.283 ± 0.1*° |
| apoE^{-/-} 1 mo | 0.337 ± 0.06 | 0.323 ± 0.04 | 0.349 ± 0.05 | 0.484 ± 0.1* | 0.493 ± 0.08* |
| apoE^{-/-} 12 mo | 0.104 ± 0.01° | 0.109 ± 0.07° | 0.09 ± 0.01° | 0.169 ± 0.05*° | 0.163 ± 0.1*° |

Values are mean ± SD of absorbance units (OD) at 450/595 nm. Periepidydimal adipose tissue was harvested from C57 or ApoE^{-/-} mice, 1 month and 12 months old. Adipose tissue-derived mesenchymal stromal cells (AT-MSCs, 2 × 10⁵ cells/mL) at passage 3, nontransduced or transduced with mock vector, or pLenti-TERT or pLenti-MYOCD vectors were plated in 96 well plate and media were replaced with serum-free media containing 10 μM BrdU at 24 h before harvesting cells.
N = 5 mice/group.
°p < 0.01, versus age 1 month;
*p < 0.05, versus mock-transduced.
Legend:
ApoE^{-/-}, apolipoprotein E deficient,
Nt, nontransduced;
MYOCD, transduced with pLenti-myocardin vector;
TERT, transduced with pLenti-telomerase reverse transcriptase vector.

TABLE 3
The effect of TERT and MYOCD transduction on cell death and
apoptosis of murine bone marrow-mesenchymal stromal cells

| | Mock | MYOCD | TERT |
|---|---|---|---|
| Fas-noninduced | | | |
| % dead cells | 22 ± 3 | 17 ± 3 | 13 ± 1 |
| % necrotic cells | 4 ± 0.2 | 38 ± 2* | 36 ± 3* |
| % apoptotic cells | 96 ± 11 | 62 ± 7* | 65 ± 6* |
| Fas-induced | | | |
| % dead cells | 37 ± 7 | 8 ± 1** | 4 ± 0.3** |
| % necrotic cells | 3 ± 0.1 | 56 ± 7** | 82 ± 13** |
| % apoptotic cells | 97 ± 17 | 39 ± 4* | 10 ± 1** |

Values are mean ± SD of percentage of positive cells for Annexin V and/or propidium iodide.
N = 5 mice/group. *p < 0.05
**p < 0.01 versus mock-transduced. Bone marrow was harvested from C57 mice, 12 months old. Bone marrow-derived mesenchymal stromal cells (BM-MSCs) (1 × 10⁶ cells/mL) at passage 3, nontransduced or transduced with mock vector or pLenti-TERT or pLenti-MYOCD vectors, were plated in 60 mm culture dishes and treated overnight with CD95/Fas (500 ng/mL).
Legend:
Mock, cells transduced with mock vector,
MYOCD, cells transduced with myocardin vector,
TERT, cells trans-duced with telomerase reverse transcriptase vector.

TABLE 4
Quantitative analysis of arteriole density in the limbs of
ApoE^{-/-} mice after treatments.

| | Arterioles (vessels/mm²) |
|---|---|
| No ligation | |
| PBS | 20 ± 2 |
| Ligation | |
| 1 day post-PBS | 7 ± 3** |
| 15 days post-PBS | 10 ± 4* |
| 15 days post-mock/MSCs | 13 ± 5* |
| 15 days post-TERT/MYOCD-GFP⁺MSCs | 17 ± 4* |

Summary of quantitative analysis of arteriole density from nonischemic limbs or limbs with femoral artery ligation of ApoE^{-/-} mice (12 months old), treated with multiple injections of phosphate buffered saline (PBS. 500 μl), or mock-transduced GFP⁺MSCs (3 × 10⁶ cells/500 μl), or TERT/MYOCD-transduced GFP⁺MSCs (3 × 10⁶ cells/500 μl). Data are presented as mean ± SD, with n = 5 mice for each treatment.
**p < 0.01, versus nonischemic limbs;
*p < 0.05, versus 1 day post-ligation.
Legend:
MSCs, adipose tissue-derived mesenchymal stromal cells;
TERT/MYOCD, co-transduced with pLenti-TERT and pLenti-myocardin vector
TERT, telomerase reverse transcripase;
MYOCD, myocardin
GFP, green fluorescence protein.

TABLE 5
Retention rate of GFP⁺ MSCs in the ischemic muscles after injection

| Treatment | Cell number |
|---|---|
| PBS, 500 μl | 0 ± 0 |
| Mock-transduced GFP⁺MSCs | 220 ± 87 |
| TERT/MYOCD GFP⁺MSCs | 420 ± 120* |

Evaluation of the number of GFP⁺MSCs in the injected tissues at postoperative days 21 were performed by counting GFP positive/bisbenzimide positive cells. An average of 20 fields in each section were examined and photographed. Values are mean ± SD for n = 5 mice for each treatment group. Control muscles, injected with PBS, showed no positive staining.
*p < 0.05 versus mock-trasnduced GFP+ MSCs.
Legend:
PBS, phosphate buffered saline;
GFP, green fluorescence protein;
MSC, adipose tissue-derived mesenchymal stromal cells;
TERT, telomerase reverse transcriptase;
MYOCD, myocardin.

1. (canceled)

2. A composition for generating mammalian stem cells from aging, damaged and/or dysfunctional mammalian cells, wherein said composition comprises:
rejuvenating senescent mesenchymal stromal cells (MSCs) from a mammalian tissue stroma wherein the rejuvenating MSCs comprise:
positive expression of telomerase, or a catalytic unit of Telomerase Reverse Transcriptase (TERT); and Myocardin (MYCOD);
positive expression of CD29+, CD44+, and CD90+, and receptors for lipoproteins in said rejuvenating MSCs;
negative or diminishing expression of CD45, and receptors for acetylated low density lipoprotein (LDL), wherein said rejuvenating senescent MSC's increase at least one of: cell regeneration, cell repair, and cell rejuvenation of said damaged cells compared to native MSC's.

3. The composition of claim 2, wherein said mammalian stem cells are multipotent, and wherein said rejuvenating cells possess a multi-potency of growth and differentiation into various cell lineages.

4. A method of making a composition for use in the rejuvenation of aging, damaged and dysfunctional mesenchymal cells, said method comprises:
isolating a mesenchymal stromal cell (MSC) from an aging, or dysfunctional tissue stroma, wherein said isolated MSCs express low or no TERT and/or MYCOD, and cannot proliferate and differentiate into different mammalian cell lineages in an apolipoprotein-containing medium;
synthesizing a nuclear acid or cDNA by reverse transcription of mRNA with oligonucleotide primers specific for TERT and MYCOD genes, wherein the cDNA comprises sequences encoding full or a partial length of coding sequences of both TERT and MYCOD genes;
constructing a expression vector carrying TERT and/or MYCOD cDNA inserts, wherein said TERT/MYCOD-carrying vector comprises a TERT gene and a MYCOD gene; and
transducing said isolated MSCs with a TERT/MYCOD gene-carrying vector; and
forming a rejuvenating MSC, wherein said rejuvenating MSC comprises increased expression of TERT and MYCOD, and thereby comprises therapeutic efficacy for regenerating or repairing diseased mammalian tissues or organs.

5. The method of claim 4, wherein said expression of both TERT and MYCOD is transduced in said aged, damaged and dysfunctional cells by cultivating in an apolipoprotein-rich medium containing a nuclear acid encoding TERT, MYCOD or both, and wherein said transduced MSCs possess interacting TERT and MYCOD, which synergize to rejuvenate MSCs and thereby serve as an indicator of rejuvenating activity in said cells.

6. The method of claim 4, wherein said rejuvenating MSCs comprise TERT and MYCOD co-expression and have enhanced interaction of TERT and MYCOD, forming a complex of TERT and MYCOD.

7. The method of claim 6, wherein said rejuvenating MSCs with TERT and MYCOD co-expression and interaction maintain a quiescent status, comprising longer telomeres, and the potential maturating into multi cell lineages, comprising cardiac and vascular cells, bone cells, and adipose cells.

8. The method of claim 7, wherein said rejuvenating MSCs with longer telomeres and higher promyogenic activity are resistant to senescence and apoptosis and form myogenic cytoskeletal proteins, such as cardiac and vascular actins.

9. The method of claim 4, wherein the expression vector is a viral expression vector or a plasmid.

10. A method of making a composition of rejuvenating CD29⁺/CD44⁺/CD90⁺ MSCs, wherein said MSCs are cultivated and harvested in an apolipoprotein-rich medium, said method comprising:
treating CD29⁺/CD44⁺/CD90⁺ MSCs from adult mammalian tissues with TERT/MYCOD cDNA in a medium comprising ApoAI, ApoB, ApoE and ApoJ, to form TERT/MYCOD-expressing CD29⁺/CD44⁺/CD90⁺ MSCs;
isolating, and harvesting said isolated TERT/MYCOD-expressing CD29⁺/CD44⁺/CD90⁺ MSCs to form harvested MSCs, wherein said harvested MSCs comprise undifferentiating, non-senescent MSCs; and
separating non-senescent TERT+/MYCOD CD29⁺/CD44⁺/CD90⁺ MSCs from senescent CD29⁺/CD44⁺/CD90⁺ MSCs by passage selection, thereby forming a composition of rejuvenating CD29⁺/CD44⁺/CD90⁺ MSCs.

11. The method of claim 10, wherein said rejuvenating CD29⁺/CD44⁺/CD90⁺ MSCs from adult adipose tissues are induced in vitro by transducing of TERT/MYCOD to form precursors of cardiac myocytes and vascular smooth muscle cells.

12. The method of claim 11, wherein transducing further comprises incubating said rejuvenating senescent CD29⁺/CD44⁺/CD90⁺ MSCs with media containing said TERT/MYCOD-carrying vector.

13. The method of claim 10, further comprising propagating said rejuvenating CD29⁺/CD44⁺/CD90⁺ MSCs by culturing in a culture medium comprising a pool of lipoproteins.

14. A method of delivering said rejuvenating MSCs of claim 2, into a mammalian subject in need of regenerating, rejuvenating or repairing at least one of cells, tissues and organs, said method comprising:
examining said subject for markers for the senescence of CD29+/CD44+/CD90+ MSCs;
determining expression and interaction of TERT and MYCOD, and telomere length and promyogenic activity; and
treating said subject in need of rejuvenating, regenerating or repairing at least one of cells, tissues and organs, by administering said rejuvenating MSCs.

15. The method of claim 14, wherein said cells, tissues or organs have been damaged by a disease.

16. The method of claim 15, wherein said disease is selected from the group consisting of at least one of tissues and organs damaged by a pathological condition, comprising chemical and mechanical injury, aging, and shortage of blood supplies (ischemia), such as atherosclerotic vascular disorders, and coronary and cerebral infarction.

17. The method of claim 14, wherein said administering said rejuvenating MSCs positive in expression of anti-senescent biomarkers and TERTIMYOCD comprises at least one of the follows: tissue injection, intravenous injection, and delivering through a catheter.

18. The method of claim 14, wherein injecting said rejuvenating MSCs are suspended in a buffer comprises at least 1-3×10⁶ rejuvenating senescent CD29⁺/CD44⁺/CD90⁺ MSCs supplemented with lipoproteins, comprising the apolipoproteins ApoAl, ApoE, and ApoJ.

19. The method of claim 14, wherein said administering of said rejuvenating senescent CD29⁺/CD44⁺/CD90⁺ MSCs further increases blood flow and revascularization of said cells, tissues, or organs.

20. The method of claim 14, wherein said rejuvenating CD29+/CD44+/CD90+ mesenchymal stromal cells (MSCs) further comprise increasing resistance to Fas-induced and Non-Fas induced apoptosis, as compared to native MSC's.

21. The method of claim 14, wherein said rejuvenating CD29⁺/CD44⁺/CD90⁺ MSCs to further develop into mesenchymal cell lineages comprising cardiac myocytes, smooth muscle cells, endothelial cells, osteoblasts, chondrocytes and adipocytes.
